SEQUENCE LISTING (6 sequences)

SEQ ID NO: 1 - protein, 4 aa, Homo sapiens
GEEG

SEQ ID NO: 2 - protein, 4 aa, Artificial
  Amino acid sequence motif
GAAG

SEQ ID NO: 3 - protein, 13 aa, Artificial
  Synthetic peptide
IQEGAAGRPKDDR

SEQ ID NO: 4 - protein, 13 aa, Artificial
  Synthetic peptide
RDDKPRGAAGEQI

SEQ ID NO: 5 - protein, 13 aa, Homo sapiens
IQEGEEGRPKDDR

SEQ ID NO: 6 - protein, 13 aa, Artificial
  Synthetic peptide
RDDKPRGEEGEQI

(19) United States
(12) Patent Application Publication
(10) Pub. No.: US 2008/0280833 A1
(43) Pub. Date: Nov. 13, 2008

(54) Therapeutic Peptides Derived from Urokinase Plasminogen Activator Receptor

(76) Inventors: Bernard Degryse, Milan (IT); Massimo Resnati, Milan (IT); Ralf-Peter Czekay, La Jolla, CA (US); David J. Loskutoff, La Jolla, CA (US); Francesco Blasi, Milan (IT)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 11/795,351

(22) PCT Filed: Jan. 31, 2006
(86) PCT No.: PCT/IB2006/000422

§ 371 Date: May 19, 2008

Related U.S. Application Data
(60) Provisional application No. 60/648,626, filed on Jan. 31, 2005.

(51) Int. Cl. A61K 38/00; C07K 7/00; C12N 15/11; C07K 16/18; A61P 31/00; G01N 33/00; C12N 15/00

(52) U.S. Cl. 514/14; 530/330; 530/327; 536/23.1; 435/320.1; 514/18; 436/86; 530/387.9

(57) ABSTRACT

The present invention relates to a chemotactically active epitope derivable from the urokinase receptor, a mutated peptide that acts as an inhibitor of chemotaxis, and their therapeutic use.

### FIELD OF THE INVENTION

The present invention relates to a chemotactically active epitope derivable from the urokinase receptor, a mutated peptide that acts as an inhibitor of chemotaxis, and their therapeutic use.

### BACKGROUND OF THE INVENTION

Cell migration and invasion are important processes in many patho/physiological conditions, such as tumor invasion, immune response to, for example, infectious agents such as HIV, angiogenesis and inflammation.

WO2005/067650 discloses a method of monitoring the response of a patient being treated for cancer by administering an anti-cancer agent which is an inhibitor of PDGRF-beta and in particular one of more polypeptides disclosed in SEQ ID Nos: 21-40. However, this sequence contains a GEEG motif which we have determined to be chemotactic and would therefore not be appropriate for the treatment of cancer.

WO2005/048822 relates to preventing cell migration using an antibody which binds to the amino terminal fragment of urokinase. It teaches that “the important role of uPA-uPAR in tumor growth and its abundant expression within tumor, but not normal tissue, makes this system an attractive diagnostic and therapeutic target”. However, crucially there is no indication of the relevant motif.

WO2004/099780 relates to a method of montioring the presence or stage of cancer comprising detecting at least one form of uPAR. However, the forms of uPAR disclosed are intact uPAR, uPAR domain 2+3 and uPAR domain 1. No recognition is given to the importance of domain 2 let alone the motif GEEG.

WO2004/007672 discloses many different Ly-6 like polypeptides for alleged use in a number of disease states.

WO03/033009 discloses a method of treating arthritis comprising the use of an inhibitor of uPA or uPAR.

DE10117381A discloses antibodies to specific uPAR variants.

U.S. Pat. No. 6,113,897 also discloses antibodies to uPAR.

U.S. Pat. No. 5,891,664 relates to the production of uPAR or parts thereof and uPAR binding uPA molecules for use as a therapeutic or diagnostic component. They allege that any polypeptide comprising at least 5 amino acids and up to the complete sequence of uPAR from amino acids 1 to 313 would be effective. There is no specific disclosure or indication of the usefulness of the GEEG motif.

U.S. Pat. No. 5,519,120 also discloses an antibody to uPAR.

WO90/12091 discusses pure UPAR and truncated forms thereof, and analogues of uPA.

EP1122318A relates to diagnostic methods for the detection of polymorphisms relates to uPAR.

US2003/0027981 discloses a polypeptide presenting an epitope cross-reactive with an epitope of uPAR. There is no specific disclosure of the GEEG motif.

U.S. Pat. No. 6,248,712 discloses a polypeptide presenting an epitope cross-reactive with an epitope of uPAR. There is no specific disclosure of the GEEG motif.

WO2005/009350 relates to compositions which modulate the movement of cells with migratory capacity.

US2003/0180302 relates to methods of promoting wound healing.

WO03/018754 relates to neural regeneration peptides and methods for their use in the treatment of brain damage.

US2003/0022835 relates to polypeptides which may be expressed in skin cells.

However, there is a continuing need to provide ways of controlling chemotactic processes. The present invention seeks to achieve this. We have also found a new way of stimulating stem cells.

### SUMMARY OF THE INVENTION

We have identified the amino acid motif GEEG (G=Gly=Glycine, E=Glu=Glutamic acid) (also herein referred to as an epitope) from human uPAR which acts as a chemotactic peptide.

Particularly, but not exclusively, peptide D2A corresponds to the original human uPAR sequence (aa130-142) and acts as chemotactic peptide. Peptide D2B has the reverse sequence of D2A and also acts as chemotactic peptide.

We have also identified the amino acid motif GAAG (A=Ala=Alanine) (also herein referred to as an epitope) which acts as an inhibitor of chemotaxis. We have found that this motif acts as a general inhibitor of chemotaxis.

Particularly, but not exclusively, peptide D2A-Ala has the same sequence as D2A, but two glutamic acids have been mutated to alanine. It is a new inhibitor of cell migration that, together with other peptides comprising the GAAG motif may be extremely useful for research purposes. In addition, such inhibitory peptides may act as or be used as a tool to develop a new drug against, for example, angiogenesis, inflammation, infectious diseases, autoimmune diseases, vascular diseases, and cancers.

### STATEMENTS OF THE INVENTION

According to one aspect of the present invention there is provided a polypeptide which inhibits cell migration, including chemotaxis, cell adhesion, cell proliferation and/or cell differentiation and which comprises the amino acid motif GAAG.

Plasminogen activators, their inhibitors, and their cell-surface receptor(s) play central roles in these processes by regulating extracellular proteolysis, cell adhesion, and signal transduction. In tissues, extracellular proteolysis is controlled by the production of plasmin that is generated by plasminogen activators, mainly urokinase (uPA)1, which binds to a specific membrane receptor, uPAR.

Fully processed human uPAR is a 45-55-kDa glycoprotein linked to the outer membrane leaflet by a glycosylphosphatidylinositol lipid anchor. The protein is composed of three homologous domains with a disulfide bonding pattern characteristic of the uPAR/Ly-6 superfamily.

Besides providing the cells with the means to perform directed extracellular matrix degradation, binding of uPA to uPAR has profound effects on cell adhesion, migration, and proliferation.

We have found that the GAAG motif interacts with such uPA-mediated processes.

Although binding to uPAR seems to be required, adhesion, migration, proliferation and/or differentiation are often independent of the proteolytic activity of uPA, strongly suggesting that other protein interactions are involved. For example, uPAR has the ability to bind vitronectin (Vn), a function that has been shown to induce cell adhesion and to change gene expression during the differentiation of human myeloid U937 cells.

We have found that the GAAG motif interacts with vitronectin (VN)-mediated cell processes, including adhesion, migration, proliferation and/or differentiation.

We have found that the GAAG motif interacts with integrin-mediated signalling and thus affects cell adhesion, migration, proliferation and/or differentiation. For example, we have demonstrated that the GAAG motif affects αvβ3, α5β1, and/or α3β1 integrin-mediated signalling.

Fibronectin and laminin have been found to affect chemotaxis. We have also found that the GAAG motif affects fibronectin (FN)-mediated cell migration and/or laminin (LN)-mediated cell adhesion, migration, proliferation and/or differentiation.

Epidermal growth factor is a potent chemoattractant. We have found that the GAAG motif interacts with EGF-mediated cell adhesion, migration, proliferation and/or differentiation and/or UTP-mediated cell adhesion, migration, proliferation and/or differentiation.

We have also found that the GAAG motif inhibits insulin-mediated cell adhesion, migration, proliferation and/or differentiation.

According to one embodiment of the present invention there is provided a polypeptide comprising the amino acid motif GAAG and which polypeptide is obtainable or derivable from the urokinase receptor (uPAR).

In another embodiment, the polypeptide consists of the amino acid motif GAAG and which polypeptide is derivable from the urokinase receptor (uPAR).

In one embodiment the polypeptide is not SEQ ID NO: 1 or 2 from EP1122318.

Preferably, the polypeptide comprises or consists of the amino acid sequence IQEGAAGRPKDDR or RDDKPRGAAGEQI.

According to another embodiment the polypeptide comprises or consists of domain 2 of uPAR in which the amino acid residues at positions 34 and 35 of the wild type sequence are changed from glutamic acid to alanine or a fragment thereof.

According to another aspect of the present invention there is provided a polynucleotide encoding for the said polypeptide of the invention.

According to another aspect of the present invention there is provided an expression vector comprising the said polynucleotide of the present invention.

According to another aspect of the present invention there is provided a cell comprising the said expression vector of the present invention.

According to another aspect of the present invention there is provided a pharmaceutical composition comprising the polypeptide, the polynucleotide, the expression vector, or the cell of the present invention, together with a pharmaceutically acceptable carrier, excipient or diluent.

According to another aspect of the present invention there is provided a method for treating or controlling angiogenesis, fibrosis of tissue, inflammation, cancer, an immune disorder, epithelial cell hyperplasia, an infectious disease or a disease associated therewith comprising administering an effective amount of the polypeptide, the polynucleotide, the expression vector, the cell, or the pharmaceutical composition of the present invention to a patient in need of the same.

According to another aspect of the present invention there is provided a polypeptide which activates cell migration, including chemotaxis, cell adhesion, proliferation and/or differentiation and which comprises the amino acid motif GEEG. Such processes may be activated by any of the above mentioned pathways.

In one embodiment the peptide comprising the amino acid motif GEEG is obtainable or derivable from the urokinase receptor (uPAR).

In other embodiments the polypeptide is not:

SEQ ID NO: 35 of WO2005/067650, and/or
SEQ ID NO: 1 of WO2005/048822, or amino acids 49-135 thereof, and/or
naturally occurring uPAR, uPAR domain 1, 2 or 3 or a combination thereof, in particular uPAR domains 2+3, and/or
SEQUENCE IS NO: 102 of WO2004/007672, and/or
SEQUENCE IS NO: 6 of WO03/033009 (GenBank Q03405), and/or
SEQUENCE IS NO: 8 of DE10117381A, and/or
the uPAR sequence disclosed in U.S. Pat. No. 6,113,897 or U.S. Pat. No. 5,891,664, and/or
the uPAR sequence disclosed in SEQ ID NO:4 of U.S. Pat. No. 5,891,664, and/or
the uPAR sequence disclosed in SEQ ID NO:4, 12 or 13 of U.S. Pat. No. 5,519,120, and/or
a pure form of uPAR as disclosed in WO90/12091, nor is it the receptor binding domain of the 15 kD amino-terminal fragment of uPA, and/or
the uPAR-binding sequence disclosed in claim 1 of US2003/0027981 and in particular SEQ ID NO:3 thereof, and/or an amino acid sequence derived from a u-PAR which polypeptide comprises at least 5 amino acids and up to the complete sequence of u-PAR, particularly when said sequence does not contain the sequence GEEG, and/or
the sequence disclosed in SEQ ID NOS: 4, 22 or 23 of U.S. Pat. No. 6,248,712, and/or a heat shock protein and more preferably not HSP60 of WO2005/009350 and/or the peptide disclosed in J Chem Soc, Perkin Transactions 1:Organic and Bio-Organic Chemistry (1994), vol. 21, pages 3201-7, and/or
SEQ ID NO:31 or 32 of WO2003/0180302, and/or
SEQ ID NO:22 of WO03/018754, and/or
SEQ ID NO: 138, 294 or 382 of US 2003/0022835, including any combination of the aforementioned sequences.

In one embodiment, the polypeptide consists of the amino acid motif GEEG and which polypeptide is derivable from the urokinase receptor (uPAR).

In another embodiment, the said polypeptide comprises or consists of the amino acid sequence IQEGEEGRPKDDR or the amino acid sequence RDDKPRGEEGEQI.

According to another aspect of the present invention there is provided a polypeptide comprising or consisting of the amino acid sequence of domain 2 of the uPAR or its reverse or a fragment thereof.

According to another aspect of the present invention there is provided a polynucleotide encoding for the said polypeptide of the invention.

According to another aspect of the present invention there is provided an expression vector comprising the said polynucleotide of the present invention.

According to another aspect of the present invention there is provided a cell comprising the said expression vector of the present invention.

According to another aspect of the present invention there is provided an antibody directed against any of the polypeptides of the present invention.

According to another aspect of the present invention there is provided a pharmaceutical composition comprising the aforementioned polypeptide, the polynucleotide, the expression vector, the cell, or the antibody of the present invention, or a polypeptide derivable from uPAR and which lacks domain 2 thereof, together with a pharmaceutically acceptable carrier, excipient or diluent.

According to another aspect of the present invention there is provided a method for treating a disease associated with a reduction in stem cell mobilization comprising administering an effective amount of the aforementioned polypeptide, the polynucleotide, the expression vector, the cell, or the pharmaceutical composition of the present invention to a patient in need of the same.

According to yet another aspect of the present invention there is provided a use of the aforementioned polypeptide, the polynucleotide, the expression vector, the cell, or the pharmaceutical composition of the present invention as an adjuvant in a stem cell transplant.

According to a further aspect of the present invention there is provided a method of identifying an agent that is a modulator of uPAR comprising: determining uPAR activity in the presence and absence of said agent; comparing the activities observed; and identifying said agent as a modulator by the observed differences in uPAR activity in the presence and absence of said agent; and wherein the method involves the use of any of the polypeptides of the present invention.

According to another aspect of the present invention there is provided use of the polypeptide of the said polypeptide, polynucleotide, expression vector, cell, or pharmaceutical composition as an adjuvant in a stem cell transplant.

According to another aspect of the present invention there is provided a method of a stimulating stem cells including hematopoietic CD-34 positive stem cells, such as KG-1 stem cells, comprising applying the said polypeptide, polynucleotide, expression vector, cell, pharmaceutical composition to a cell population.

According to yet another aspect of the present invention there is provided a method of identifying an agent that is a modulator of uPAR, integrin, such as αvβ3, α3β1, α5β1, VN, FN, LN, EGF-R, P2Y2, insulin-R activity comprising: determining uPAR, integrin, such as αvβ3, α3β1, α5β1, VN, FN, LN, EGF-R, P2Y2, insulin-R activity respectively in the presence and absence of said agent; comparing the activities observed; and identifying said agent as a modulator by the observed differences in uPAR integrin, α5β1, α3β1, VN, FN, LN, EGF-R, P2Y2, insulin-R activity (as appropriate) in the presence and absence of said agent; and wherein the method involves the use of any of the polypeptides of the present invention.

In one embodiment the method further comprises preparing said agent.

According to a further aspect of the present invention there is provided an agent identifiably according to the method of the present invention or prepared according to the method of the present invention.

### DESCRIPTION OF THE FIGURES

FIGS. 1a-d are a series of graphs showing that the chemotactic activity of VN correlates with the level of uPAR expression.

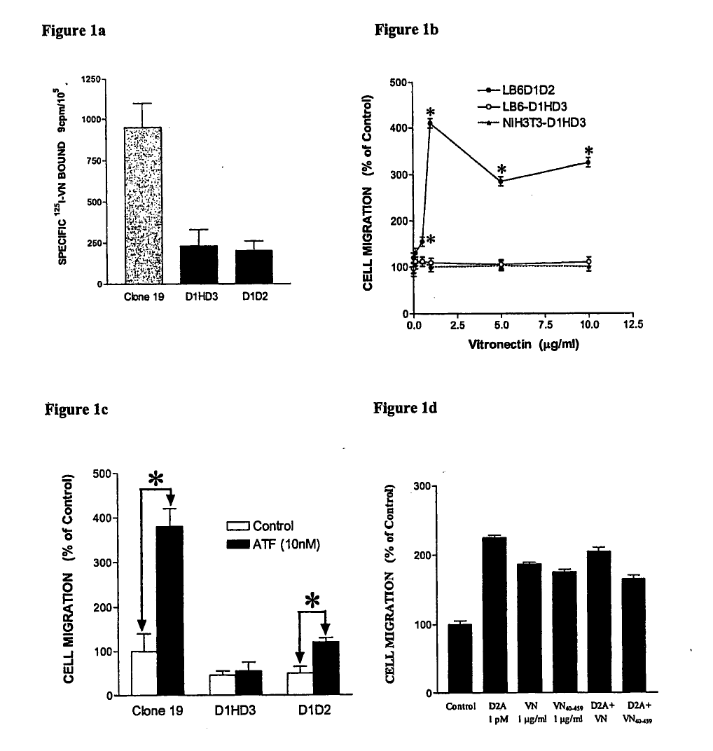

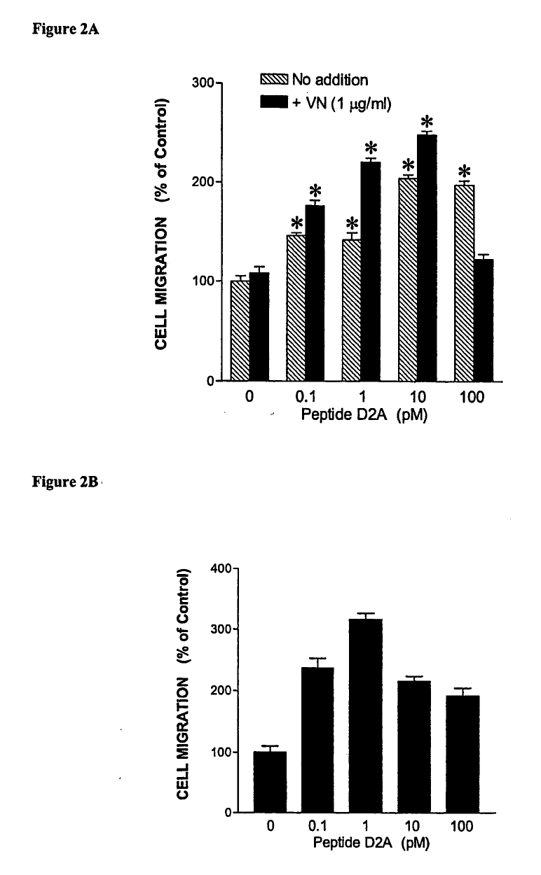

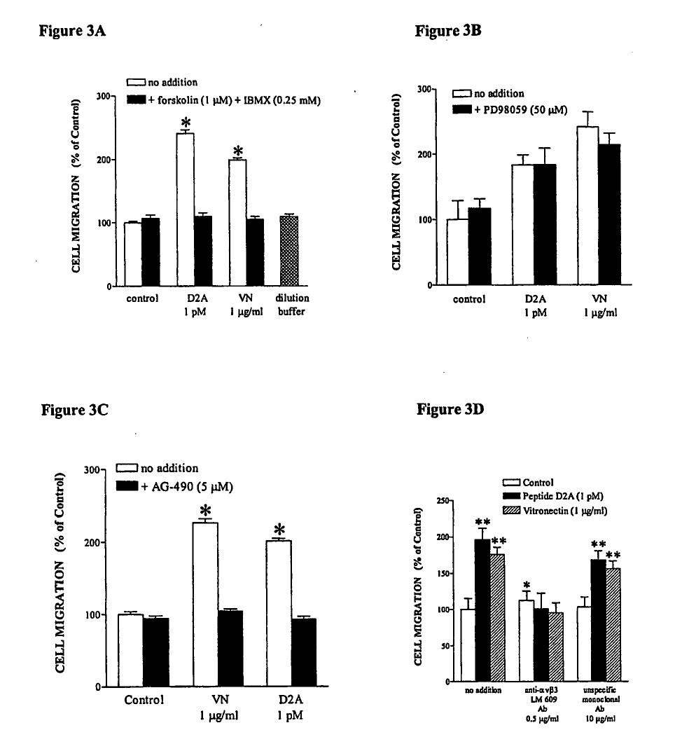

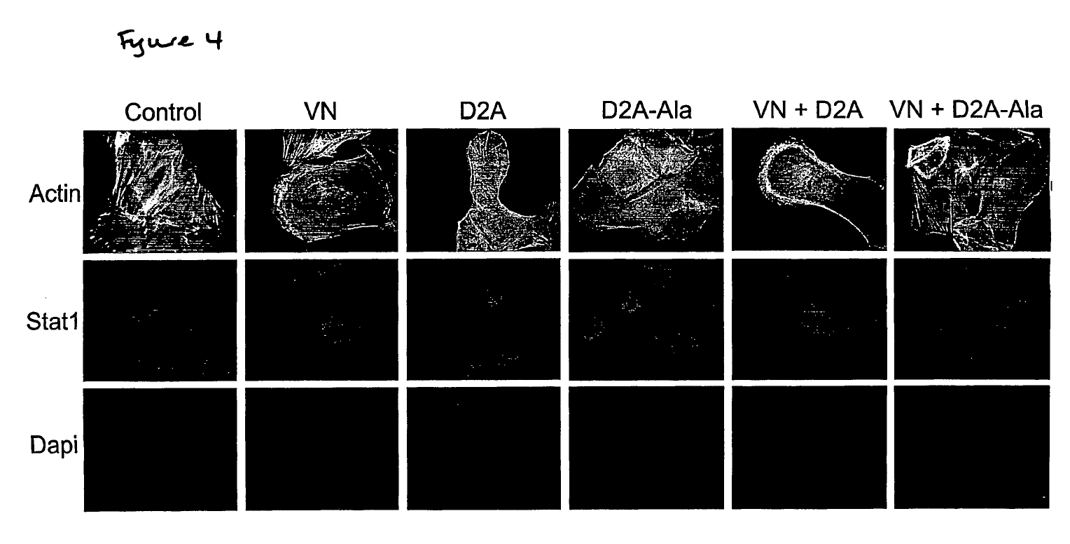

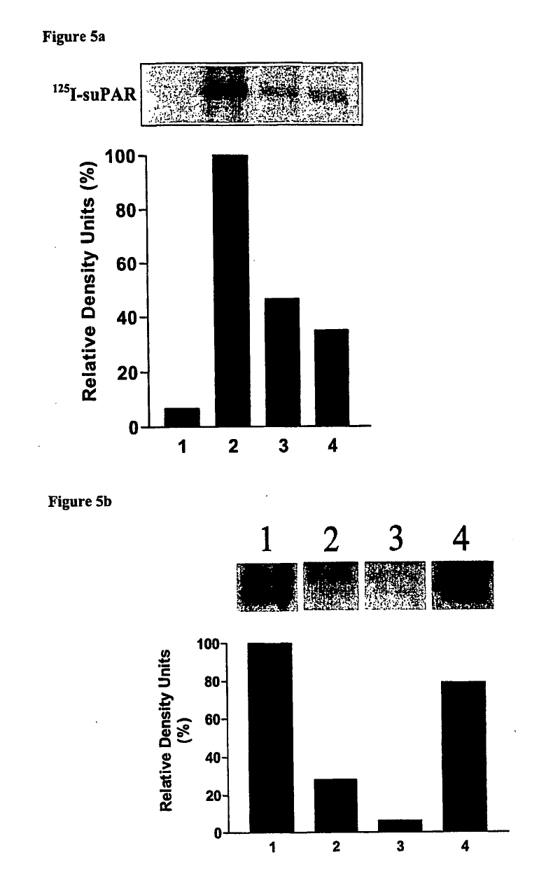

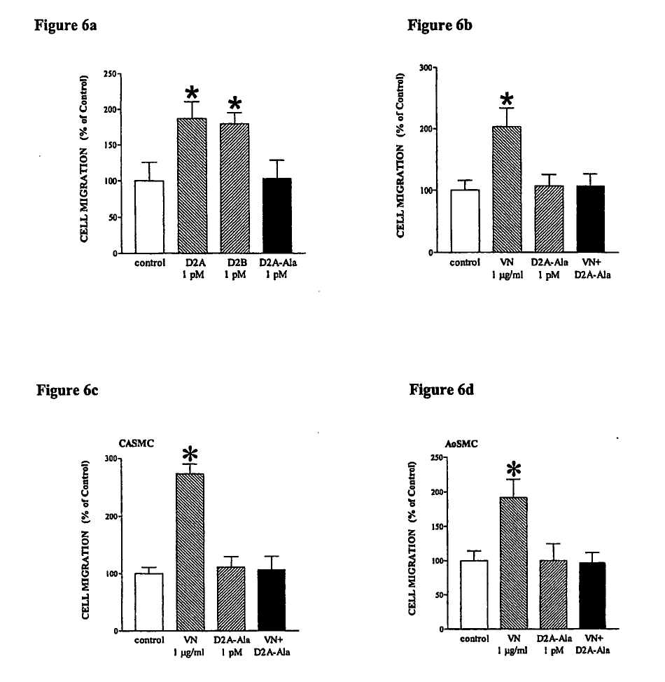

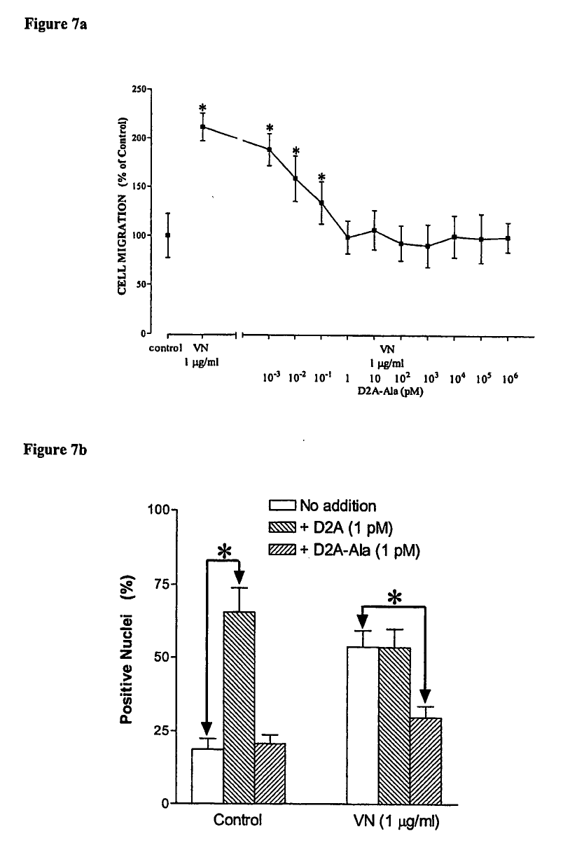

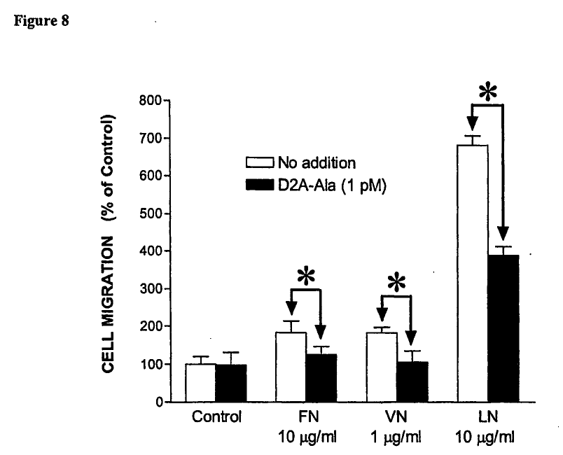

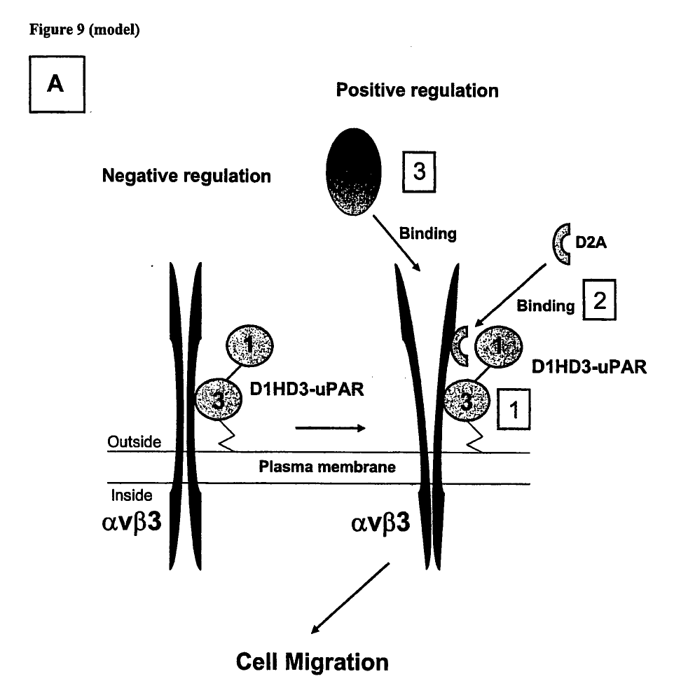

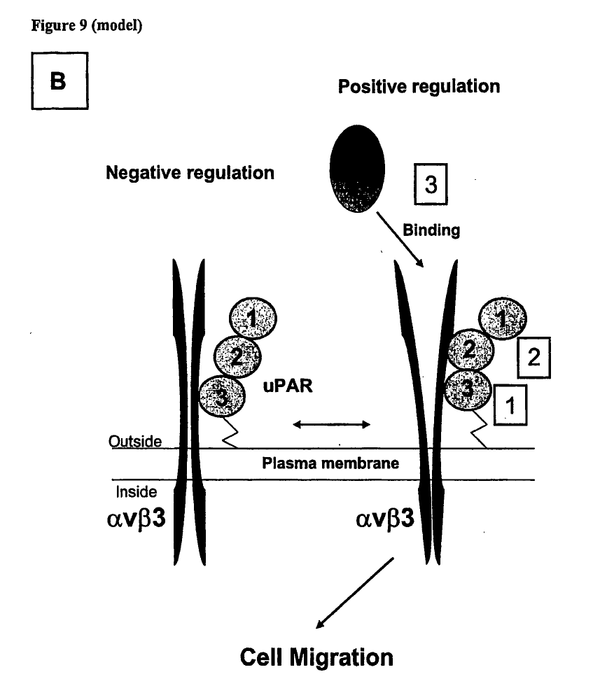

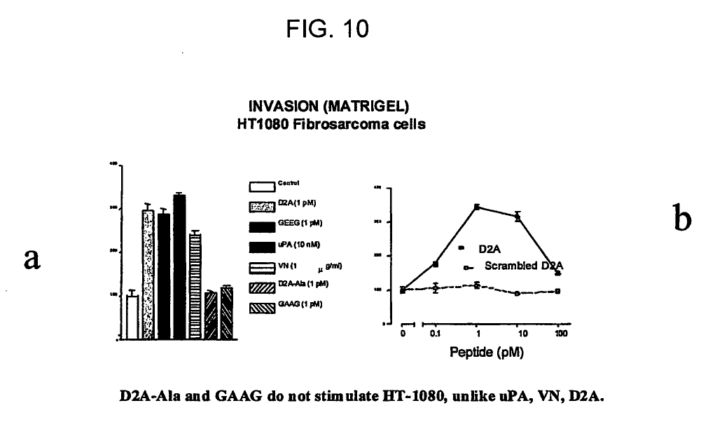

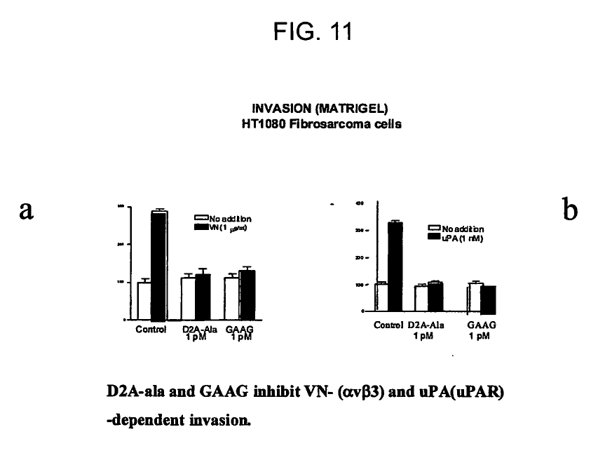

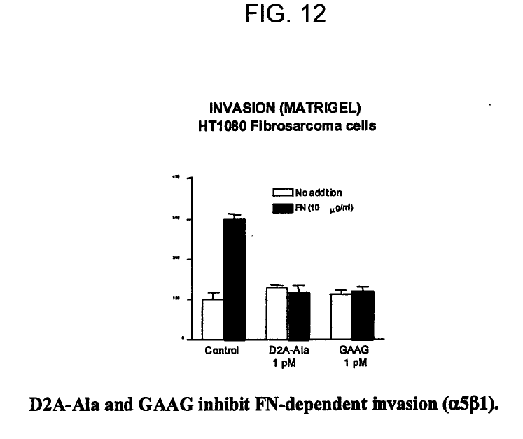

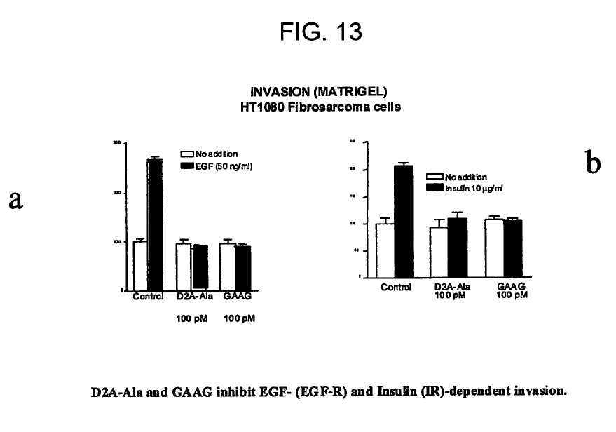

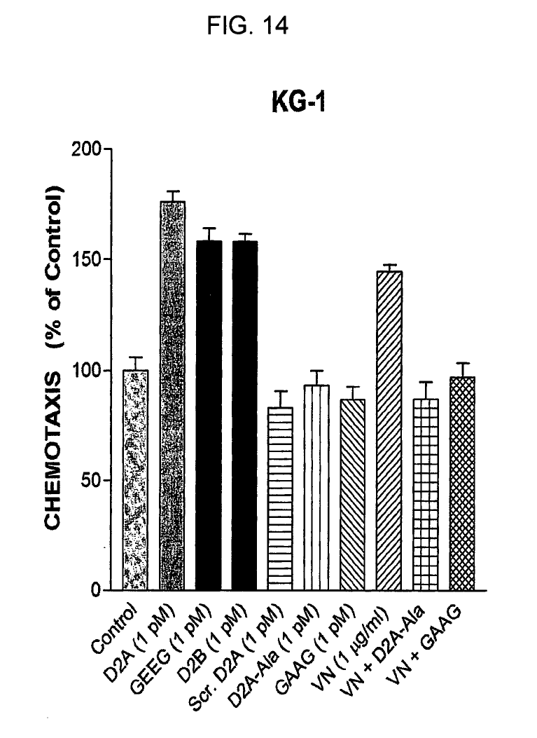

In more detail, in FIG. 1 (A) VN binding to LB6 cells transfected to express wild-type human uPAR, or a mutated form of uPAR lacking either domain 2 (LB6-D1HD3) or domain 3 (LB6-D1D2). Binding of ¹²⁵I-VN was performed as described in the Examples below. Cells were incubated with ¹²⁵I-VN (0.5 nM) on ice for 90 minutes, washed and lysed. Nonspecific binding was determined by competition with 50 nM VN and subtracted. Results are the mean ±SD from four experiments performed in triplicate. (B) Effect of increasing concentrations of VN on chemotaxis of cells expressing different forms of uPAR. LB6-D1HD3 and NIH 3T3-D1HD3 cells which express recombinant u-PAR lacking domain 2. LB6-D1D2 cells express a u-PAR form lacking domain 3. The data represent three experiments in triplicate. Random cell migration was considered 100%. The asterisk indicates statistical significance (P<0.002). (C) Effect of ATF on migration of LB6-D1D2 and LB6-D1HD3 cells compared to LB6 clone 19 cells. The asterisk indicates statistical significance (P<0.001). (D) VN-induced migratory signal did not depend upon binding of VN to uPAR. Both full-length VN and VN₄₀₋₄₅₉ lacking the SMB domain, have similar chemotactic activity on RSMC. Moreover, the addition of optimal doses of peptide D2A plus either VN or VN₄₀₋₄₅₉ does not lead to additive effects on cell migration. Peptide D2A is a synthetic peptide derived from the human sequence of domain 2 of uPAR. Chemotaxis assay was performed as described in the Examples below. All treatments increase the migratory response relative to control (P<0.0001).

FIGS. 2a-b are graphs showing that peptide D2A has a permissive effect for VN and a chemotactic activity

In more detail, in Figure (A) Peptide D2A reverts the block in VN-induced migration in LB6-D1HD3 cells. Effect of increasing doses of peptide D2A in the presence or absence of VN (1 μg/ml). The asterisk indicates statistical significance P<0.001. (B). Concentration-dependence of D2A-stimulated RSMC chemotaxis. Chemotaxis assay was performed as described in the Examples below. The asterisk indicates statistical significance (P<0.001). Migration in the absence of attractant is referred to as 100% migration.

FIGS. 3a-d are graphs showing the effect of signaling inhibitors on RSMC chemotaxis.

In more detail, in FIG. 3 (A) Both D2A and VN chemotactic effects on RSMC are completely inhibited by the addition of a combination of forskolin plus IBMX. The dilution buffer used to solubilize the mix of forskolin and IBMX has no effect. The asterisk indicates statistical significance (P<0.0001). FIG. 3(B) The MEK (MAP kinase kinase) inhibitor, PD98059, fails to block D2A- and VN-induced RSMC migration. FIG. 3 (C) AG-490, a specific inhibitor of the Jak kinases blocks both D2A- and VN-dependent chemotaxis of RSMC. The asterisk indicates statistical significance (P<0.0001). FIG. 3 (D) A. monoclonal antibody against αvβ3 (LM 609) inhibits both D2A- and VN-induced RSMC migration. (**), Difference highly significant from control (P<0.0001); (*) Significant difference (P<0.05). The 100% value represents the number of cells migrating in the absence of attractant.

FIG. 4 shows a comparison of the effects of D2A, D2A-Ala and VN on actin cytoskeleton organization, cell morphology, and the distribution of Stat1 in RSMC.

In more detail, cells were treated with either D2A, D2A-Ala (1 pM) or VN (1 μg/ml) for 30 minutes at 37° C., and then fixed, permeabilized, and triple-stained with FITC-phalloidin, Dapi, and a primary anti-Stat1 antibody followed by a secondary TRITC-anti-Igs antibody to visualize respectively the actin cytoskeleton, the nucleus, and Stat1. Untreated RSMC kept for 30 minutes at 37° C. served as control.

FIGS. 5a-b are graphs showing that peptides D2A and D2A-Ala disrupts uPAR-αvβ3 and uPAR-α5β1 complexes.

In more detail, in FIG. 5(A) ¹²⁵I-soluble uPAR was incubated in vitro for 4 hours at 4° C. with purified αvβ3 integrin in the absence (control) or in the presence of the indicated molecules. Then, complexes were immunoprecipitated with a monoclonal antibody against αvβ3 and protein G-agarose beads. Immunoprecipitated proteins were fractionated by SDS-PAGE. Gels were analyzed by autoradiography and densitometry. Data are normalized to the density of the band obtained in the presence of uPA alone (i.e. referred to as 100%), and expressed in percent of arbitrary density units. Lane 1, control (no addition); Lane 2, +uPA; Lane 3, +uPA and D2A; Lane 4, +uPA and D2A-Ala. FIG. 5 (B) Cell-free assay using αvβ1 integrin. Lane 1, control (no addition); Lane 2, +uPA; Lane 3, +uPA and D2A; Lane 4, +uPA and D2A-Ala.

FIGS. 6a-d are graphs showing that peptide D2A-Ala has no chemotactic activity but is an inhibitor of VN-induced cell migration.

In more detail, in FIG. 6 (A) Comparison of the effects of peptides D2A, D2B and D2A-Ala on migration of RSMC. (B,C,D) D2A-Ala blocks VN-induced chemotaxis of rat SMC FIG. 6 (B), and human SMC from the coronary artery FIG. 6 (C), and from the aorta FIG. 6 (D). Chemotaxis assay was performed as described in the Materials and Methods section. Migration of SMC towards medium alone is considered to be 100% migration. The asterisk indicates statistical significance (P<0.0001).

FIGS. 7a and b are graphs showing that peptide D2A-Ala is an inhibitor of both VN-induced chemotaxis and signaling.

In more detail, in FIG. 7 (A) D2A-Ala inhibits VN-induced migration of RSMC in a dose-dependent manner. Chemotaxis assay was performed as described in the Examples below in the absence (control) or in the presence of VN (1 μg/ml) with or without increasing doses of D2A-Ala. Migration of RSMC towards medium alone (control) is considered to be 100% migration. The asterisk indicates statistical significance P<0.001. FIG. 7(B) Effects of peptides D2A and D2A-Ala on the state of activation of Stat1. Cells were treated with either D2A or D2A-Ala (1 pM) in the absence or in the presence of VN (1 μg/ml) for 30 minutes at 37° C., and then fixed, permeabilized, and triple-stained with FITC-phalloidin, Dapi, and a primary anti-Stat1 antibody followed by a secondary TRITC-anti-Igs antibody to visualize respectively the actin cytoskeleton, the nucleus, and Stat1. Untreated RSMC kept for 30 minutes at 37° C. served as control. Then, random pictures were taken and nuclei positively stained for Stat1 were counted and expressed in percent. The asterisk indicates statistical significance (P<0.01).

FIG. 8 is a graph showing that peptide D2A-Ala can block integrin-dependent cell migration.

In more detail, comparison of the inhibitory effects of D2A-Ala on cell migration induced by VN, FN and LN. Chemotaxis assay was performed as described in the Example section. The value 100% represents the number of RSMC migrating in the absence of attractant. The asterisk indicates statistical significance (P<0.0001).

FIGS. 9a and b illustrates schematically a model explaining the regulatory role of uPAR on integrin function

In more detail, in FIG. 9 (A) On the left, D1HD3-uPAR has a dominant-negative effect blocking the function of the integrin, most probably by interacting with αvβ3 through site(s) located in its domain 3. On the right, peptide D2A may be the trigger that unlocks the integrin. D2A harbors the sequence of uPAR that induces signaling and cell migration by binding to αvβ3, and thus can rescue the blocking provoked by D1HD3-uPAR. FIG. 9 (B) On the left, only site(s) located in the domain 3 of uPAR interacts with αvβ3 leading to a negative regulation of the integrin. In this situation, uPAR has a particular conformation that we called d3. On the right, the sequence of uPAR harbored by peptide D2A is the switch that leads to the positive regulation of integrin function. 1. “Anchorage” site(s) located on domain 3 interact with the integrin; 2. D2A sequence binds to αvβ3 positively regulating its function, in this case uPAR is in another conformation that we named d3d2; 3. VN can bind to the integrin.

FIG. 10, Panels a and b are graphs showing that D2A-Ala and GAAG do not stimulate HT-1080, unlike uPA, VN and D2A.

FIG. 11, Panels a and b are graphs showing that D2A-Ala and GAAG inhibit VN-(αvβ3) and uPA (UPAR)-dependent invasion.

FIG. 12 is a graph showing that D2A-Ala and GAAG inhibit FN-dependent invasion (α5β1).

FIG. 13, Panels a and b are graphs showing that D2A-Ala and GAAG inhibit EGF— (EGF-R) and insulin (IR)-dependent invasion.

FIG. 14 is a graph showing that D2A and GEEG stimulated migration of KG-1 cells.

### DETAILED DESCRIPTION OF THE INVENTION

Various preferred features and embodiments of the present invention will now be described by way of non-limiting example.

The practice of the present invention will employ, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O'D. McGee, 1990, In Situ Hybridization: Principles and Practice; Oxford University Press; M. J. Gait (Editor), 1984, Oligonucleotide Synthesis: A Practical Approach, Irl Press; and, D. M. J. Lilley and J. E. Dahlberg, 1992, Methods of Enzymology: DNA Structure Part A: Synthesis and Physical Analysis of DNA Methods in Enzymology, Academic Press. Each of these general texts is herein incorporated by reference.

### Cell Migration, Adhesion Proliferation and Differentiation

Cell migration accompanies us from conception to death. This integrated process is initially involved in the morphogenesis of the embryo during development. The failure of cells to migrate or migration of cells to inappropriate locations can result in life threatening consequences, such as the congenital defects prominent in the brain. In the adult, cell migration, is central to homeostatic processes such as mounting an effective immune response and the repair of injured tissues. It contributes to pathologies including vascular disease, chronic inflammatory diseases, and tumor formation and metastasis. The present invention finds application in all these areas.

Defects in the migration proteins involved in development can result in malformed embryos, where tissues are disorganized because their component cells have failed to travel to their appropriate location or despite having traveled appropriately they fail to form the appropriate connections with neighboring cells and their surroundings. Those defects that do not result in early fetal death can lead to a number of congenital abnormalities in brain development resulting in epilepsy, focal neurological deficits and mental retardation.

Immunity and wound healing are two homeostatic processes in the body that rely on the ability of cells to migrate.

In addition when problems arise in these cell migratory processes, pathologies develop. When immune responses continue indefinitely, they result in chronic inflammatory conditions where migration related proteins appear to play a pivotal role. Asthma is a chronic inflammation of the airways resulting from an ongoing immune response to foreign materials (allergens) inhaled from the environment. The constant presence and activation of white blood cells in the airways (lungs) of asthmatics causes tissue damage resulting in hyper-reactivity of the airways to otherwise innocuous stimuli such as exercise, stress and cold air. In rheumatoid arthritis, the constant destruction of joint tissue by inflammatory cells migrating into these compartments as part of an autoimmune disorder, results in compromised limb function and crippling pain.

In outline, migration is a dynamic, cyclical process in which a cell extends a protrusion at its front, which in turn attaches to the substratum on which the cell is migrating. This is followed by a contraction that moves the cell body forward toward the protrusion, and finally the attachments at the cell rear release as the cell continues to move forward. The cycle is initiated by external signals (chemotactic molecules), which are sensed and communicated to the cell's interior by specialized receptive proteins in the cell membrane. In response to these signals, cells extend protrusions, by polymerizing actin, that act as feelers, seeking out new terrain and sensing the direction from which they are receiving signals. Once the direction for movement is established the machinery for enabling movement assembles with regard for the direction of migration. Adhesive complexes needed for traction collect at the front of the protrusion, tethering the protrusion to the substratum. Actomyosin filaments contract at the front of the cell and pull the cell body toward the protrusion. Release of adhesive connections in the rear of the cell and retraction of the tail completes the cycle. The orchestration of this complex process resides in many molecules that serve to distinguish the front from the rear of the cell and whose actions are carefully timed.

The role of the urokinase receptor (uPAR) in the regulation of cell migration has been widely studied (for reviews see Blasi and Carmeliet, 2002; Degryse 2003). By binding urokinase (uPA), uPAR mediates a signal that induces migration of both adherent and non-adherent cells which can be either normal or tumoural. In uPAR^{-/-} -mice, neutrophil recruitment is greatly decreased in response to pulmonary Pseudomonas aeroginosa infection when compared to wild-type mice. Other studies have demonstrated that leukocyte recruitment into the site of acute inflammation is dramatically reduced in uPAR-deficient mice (Gyetko et al., 2000, 2001; Rijneveld et al., 2002; May et al., 1998). These data explain the involvement of uPAR in physiological and pathological processes requiring cell migration such as angiogenesis, tumour invasion, response to infectious diseases and inflammation. However, the effects of uPAR are not limited to the control of cell migration, since by binding uPA uPAR also regulates pericellular proteolysis localizing uPA on the cell surface and enhancing the rate of activation of plasminogen into the fully active serine protease plasmin. Furthermore, uPAR promotes cell adhesion directly by binding vitronectin (VN), a molecule from the extracellular matrix (Waltz et al., 1994; Wei et al., 1994), and high molecular kinin-free kininogen, a recently discovered ligand which competes with VN for binding to uPAR, and thus has anti-adhesive properties (Kanse et al., 1996). Indirectly uPAR may affect cell adhesion through lateral interactions with membrane proteins such as the integrins or uPARAP (Urokinase Receptor-Associated Protein) (for reviews see Chavakis et al., 2002; Ossowski and Aguirre Ghiso, 2000).

uPAR is bound to the plasma membrane by a glycosyl-phosphatidyl-inositol (GPI) anchor, and thus has no cytoplasmic domain. uPAR consists of three homologous domains. The N-terminus of the receptor, domain 1, constitutes the primary site for the binding of uPA. However, domain 3 is also directly involved in binding, and the presence of domains 2 and 3 enhances the affinity of domain 1 for uPA (Ploug, 2003). uPAR affinity for VN is also enhanced by the binding of uPA (Wei et al., 1994). In between domain 1 and 2, lays the linker region of uPAR where its minimum chemotactic epitope, a short sequence of 5 residues ₈₈SRSRY₉₂ has been identified (Resnati et al., 1996; Fazioli et al., 1997; Nguyen et al., 1998; Degryse et al., 1999). This sequence is responsible for the migratory properties of uPAR. uPA binding to uPAR induces a conformational change of the receptor that exposes this previously masked SRSRY epitope. This change switches uPAR into a ligand of FPRL1 which in turn stimulates cell migration (Resnati et al., 1996, 2002; Fazioli et al., 1997; Degryse et al., 1999). FPRL1 (or Lipoxin A4 receptor, LXA4R) is a seven-spanning membrane receptor known as the low affinity receptor for fMLP (Resnati et al., 2002) and belonging to the FPR family. Recent data indicate that also other members of the FPR family, like FPR itself and FPRL2 may transduce the signal from SRSRY (Selleri et al., 2004; De Paulis et al., 2004). Therefore, due to similarities between the mechanism of action of uPAR and chemokines, it is possible to consider uPAR as a Membrane-Anchored ChemoKINE-like proteins (MACKINE) (Degryse, 2003; Degryse and de Virgilio, 2003). Domains 2 and 3 have been reported to be the binding sites for the two-chain kinin-free high molecular weight kininogen (HKa), which binds to uPAR in Zn²⁺-dependent manner (Colman et al., 1997; Chavakis et al., 2000). HKa can compete with VN for binding to uPAR giving to uPAR interesting adhesive and anti-adhesive properties.

Despite the lack of cytoplasmic domain, uPAR is capable of complex signaling. In the membrane, uPAR is present in large complexes including for instance signaling molecules such as hck, c-Src and FAK (Focal Adhesion Kinases). Protein kinases A and C have been shown to regulate uPAR-dependent signaling pathways (Degryse et al., 2001a). Furthermore, uPA binding to uPAR activates downstream signaling pathways including the MAP kinases (Resnati et al., 1996; Nguyen et al., 1998; Degryse et al., 2001a). uPAR also controls small G proteins, like Rac (Kjoeller and Hall, 2001), that are involved in the regulation of the cell cytoskeleton (Ridley et al., 1992) and cell morphology.

As uPAR does not have a cytoplasmic domain, it must interact with other receptors. uPAR interacts with numerous molecules in the plasma membrane, for instance, FPRL1, the EGF receptor, gp130, and integrins. Moreover, uPAR has been shown to activate and/or modulate the signaling pathways induced through these receptors (Blasi and Carmeliet, 2002; Degryse 2003). We have previously shown that u-PA- and VN-induced chemotaxis, cytoskeleton reorganization, and cell shape changes required the formation of a uPAR-αvβ3 signaling complex (Degryse et al., 1999, 2001a). While several studies have identified amino acids sequences in the alpha subunit of integrins which are able to bind uPAR and hence interfere with uPAR-integrin interaction (Wei et al., 1996, 2001; Simon et al., 2000), no similar information is available for uPAR. We have now investigated the relationship between uPAR and αvβ3, and found a sequence located in the domain 2 of uPAR that plays a structural and functional role in this interaction.

### Modulators

We have investigated the nature and the function of the interaction between the urokinase receptor (uPAR) and the integrin αvβ3. Although, vitronectin (VN) does not induce cell migration by binding to uPAR, uPAR expression significantly boosts VN-induced cell migration. These results suggest that uPAR is involved in the regulation of VN/αvβ3-dependent cell migration by interacting laterally with αvβ3. In contrast to cells transfected to express full-length human uPAR, cells expressing a uPAR mutant lacking domain 2 do not migrate in response to VN challenge.

Interestingly D2A, a synthetic peptide derived from the sequence of domain 2, can overcome the effect of this mutation allowing these latter cells to respond to VN. D2A has chemotactic activity that can be inhibited by agents blocking VN/αvβ3-dependent cell migration suggesting that D2A activates αvβ3-dependent signaling pathways. Indeed, D2A disrupts uPAR-αvβ3 and uPAR-αvβ1 complexes suggesting that D2A can interact with various integrins. Replacement of two glutamic acids by two alanines generates peptide D2A-Ala which has lost chemotactic activity. Moreover, these changes turned D2A-Ala into a wide inhibitor of integrin-dependent cell migration. In conclusion, we have identified a new chemotactic sequence in domain 2 of uPAR that induces cell migration by binding to and activating αvβ3-dependent signaling pathways. This sequence seems to play a pivotal role in the regulation of uPAR-integrin interactions.

Thus, the present, invention provides modulators of cell migratory activity, as well as related activity such as cell adhesion, proliferation and/or differentiation.

Moreover we have found potent modulators of cell migration, including chemotaxis, cell adhesion, cell proliferation and/or differentiation which work through a range of different stimuli. As well as the aforementioned uPAR and integrin activity, such as αvβ3, α3β1, α5β1-dependent activity, the modulators affect activity which is dependent upon other stimuli, such a VN, FN, EGF, UTP and insulin.

The term “modulate” as used herein refers to a change or alteration in the biological activity of uPAR and other stimuli involved in cell adhesion, including chemotaxis, migration, proliferation and/or differentiation. The term “modulate” also refers to the ability of a chemical or biological agent to affect cell migration, adhesion, proliferation and/or differentiation.

“Modulation” refers to the capacity to either increase or decrease a measurable functional property of biological activity or process by at least 10%, 15%, 20%, 25%, 50%, 100% or more; such increase or decrease may be contingent on the occurrence of a specific event, and/or may be manifest only in particular cell types.

More specifically, the present invention relates to the use of compounds which will inhibit or block (antagonise) cell migration, adhesion, proliferation and/or differentiation. The present invention also relates to the use of compounds which will increase or activate (agonise) cell migration, adhesion, proliferation and/or differentiation. Such modulation may occur directly or indirectly and may involve partial modulation.

In more detail, the GPI-membrane bound uPAR that has been first known as a focus point and a key regulator of plasminogen activation is yet recognized both as a signaling and adhesive receptor. Because uPAR does not have a cytoplasmic domain, to achieve this surprising paradigm interactions with other receptors at the level of the cell membrane are required. A wide diversity of trans-membrane receptors have been reported to interact with uPAR including for instance LRP and other internalization receptors, the EGF receptor, the G protein-coupled receptor FPRL1 (for reviews see, (Blasi and Carmeliet, 2002), Degryse and Di Virgilio, 2003). In the case of FPRL1, uPA binding to uPAR induces a conformational change which exposes the chemotactic sequence located in the linker region between domain 1 and 2 of uPAR. This conformational change turns uPAR into a ligand for FPRL1, which mediates at last the chemotactic signal of uPA (Resnati et al., 1996; Fazioli et al., 1997; Degryse et al., 1999; Resnati et al., 2002). A similar mechanism might apply for the two recently discovered novel uPAR mediators, FPR and FPRL2.

Integrins are another important family of receptors interacting with uPAR (Chapman, 1997; Ossowski and Aguirre Ghiso, 2000; Preissner, 2000). Integrins are well known for their role in the regulation of cell adhesion and migration but unlike uPAR, they possess a cytoplasmic domain connected to downstream signaling molecules. Furthermore, integrins are capable of bidirectional signaling, conveying outside-in and inside-out signals. Perhaps for this reason, integrins interact with numerous membrane proteins such as IAP (Integrin Associated Protein), tetraspanins, and uPAR (review here). Indeed, uPAR has been shown to interact with many integrins of the 1, β2, and β3 subfamilies in both cis- and trans-manner, the best examples being αMβ2 (Mac-1), α3β1, α5β1, and αvβ3 (Wei et al., 1996, 2001; Simon et al., 2000; Tarui et al., 2000;). The role of these interactions has also been investigated and it has been suggested that uPAR behaves as a modulator of integrin function (Xue et al., 1994; Simon et al., 1996; Wei et al., 1996, 2001; Xue et al. 1997).

The role of uPAR in cell migration is not confined to mediating the uPA signal. In fact, it is involved also in other signals, like that of fMLP and VN (Gyetko et al. 1994; Yebra et al., 1996; Degryse et al., 2001a), or MCP-1 and RANTES (Furlan et al., 2004). In the present invention, we have investigated how uPAR is involved in VN cell migration and have explored the consequences of uPAR integrins interaction on cell signaling, cytoskeleton organization, cell morphology, and cell migration. We have confirmed that uPAR is an important regulator of integrin function. Although it is not absolutely required (see the effect of VN on HEK-293, NIH-3T3 or LB6 cells that are devoid of uPAR), uPAR overexpression considerably enhances VN-induced cell migration. The impact of uPAR presence on integrin αvβ3 is further demonstrated by the influence of two mutants of uPAR, D1D2-uPAR and D1HD3-uPAR. While the first only slightly alters the response to VN, the second cancels it altogether (Table 1; FIG. 1b). The effects of these mutants rule out the possibility that uPAR mediates VN chemotaxis by a direct binding mechanism, as neither of the two mutants can bind VN (FIG. 1) (Hoyer-Hansen et al., 1997). Indeed, VN₄₀₋₄₅₉, the mutant of VN that lacks the somatomedin B domain harboring the binding site for uPAR, promoted cell migration as well as full-length VN (FIG. 1d). Furthermore, VN can stimulate migration of HEK-293 cells that are devoid of uPAR. Therefore, as previously reported, VN stimulates cell migration through binding to its own integrin receptors, particularly αvβ3 (Yebra et al., 1996, 1999; Degryse et al., 2001a), and exploits the lateral interactions between integrin and uPAR (Wei et al., 1994).

This view is plainly supported by the identification of a uPAR peptide, herein referred to as “D2A”, located in the domain 2 having the amino acid sequence, ₁₃₀IQEGEEGRPKDDR₁₄₂, that on one hand abolishs the inhibitory effect of D1HD3-uPAR expression and on the other shows direct signaling properties identical to VN. Peptide D2A disrupts for example uPAR-αvβ3 and uPAR-α5β1 complexes, indicating that it can interact directly with a number of integrins. D2A binding also appears to be functionally relevant, since it stimulates migration of cells expressing uPAR, and even more importantly removes the inhibitory effect of D1HD3-uPAR expressing LB6 cells (FIG. 1). Like VN-, D2A-induced cell migration was completely blocked by LM609, a monoclonal antibody against αvβ3. Thus D2A not only binds to the integrin but also generates a signal through it. Our investigation of the downstream signaling pathways activated by peptide D2A fully agrees with this idea. Using previously identified inhibitors that discriminate between uPAR— and VN-dependent signaling (Degryse et al., 2001a), we found that D2A stimulates migration via VN-dependent and not uPAR-dependent signaling pathways. Indeed, on one hand, increasing intracellular cAMP using forskolin and IBMX, which has no effect on uPA-directed cell migration (Degryse et al., 2001a), totally inhibits both D2A- and VN-induced chemotaxis. On the other hand, PD98059, the MEK inhibitor which prevents MAP kinases activation and blocks uPA-induced cell migration (Nguyen et al., 2000; Degryse et al., 2001a), fails to inhibit both D2A and VN-dependent chemotaxis (FIG. 3). Both D2A and VN activate the Jak/Stat signaling pathway as observed for numerous other chemoattractants, such as chemokines (Aaronson and Horvath, 2002). Moreover, both D2A- and VN-promoted chemotaxis is blocked by AG490, an inhibitor of the Janus family of kinases, suggesting that D2A can activate at least one Jak. In fact, both D2A and VN induced Stat1 relocalization to the nucleus of RSMC (FIG. 5). Stats are the downstream effectors of Jaks, and once activated, these latent cytoplasmic transcription factors translocate into the nucleus. In addition, D2A promotes the appearance of the elongated morphology typical of motile cells (sometime also called hand-mirror shape) and the reorganization of actin cytoskeleton that plainly reflects this motile morphology (FIG. 5), indicating that beside the Jak/Stat pathway, D2A can activate other downstream signaling molecules such as small GTP-binding proteins that are known to regulate the organization of actin cytoskeleton (Ridley et al., 1992). This would be in keeping with previous observations in fibroblasts (Degryse et al., 1999; Kjoeller and Hall, 2001). Taken together, these observations show that D2A has signaling capacities, acting through for example αvβ3-dependent and not uPAR-controlled pathways, and that the Jak/Stat pathway is directly involved in the regulation of D2A-induced cell migration.

We propose that D2A contains at least two types of sequence information: binding to integrins, and activation of integrin-dependent signaling pathways involved in the regulation of cell migration. The dissociation between these two sets of properties was observed when we introduced mutations in the sequence of D2A. Our attention on a particular GEEG epitope was due to the fact that even though peptide D2B had a sequence which was the reverse of D2A, it was equally active in chemotaxis, and both peptides contain the same GEEG sequence. Thus, we modified GEEG into a GAAG epitope, and tested the chemotactic activity of this new peptide named D2A-Ala which was identical to D2A except for its two alanines instead of two glutamic residues. Peptide D2A-Ala was not chemotactic, and had no signaling activity (FIGS. 4,6,7) demonstrating that the GEEG sequence is the chemotactically active epitope harbored by peptides D2A and D2B. We also found that even if inactive, D2A-Ala could still interact with αvβ3 and α5β1, as indicated by its inhibition of the suPAR-integrin co-precipitation (FIG. 5). Strikingly, the introduction of the GAAG epitope turned D2A into a powerful inhibitor of VN-induced cell migration with an extremely low IC₅₀, about 10-20 fM (FIG. 7). In addition, D2A-Ala also succeeded in inhibiting Stat1 activation, the appearance of the motile cell morphology and actin cytoskeleton reorganization promoted by VN. Moreover, D2A-Ala inhibited migration induced by other ECM proteins such as FN and LN suggesting that it can block other integrins. Therefore, the inhibitory ability of D2A-Ala might reside in its ability to disrupt uPAR-integrin complexes such as uPAR— αvβ3 and uPAR— α5β1 complexes. Thus, the mutation introduced in the GEEG epitope of D2A destroyed its signaling information without affecting its ability to interact with αvβ3 integrin.

When investigating the effects of D2A and D2A-Ala on cells expressing no uPAR, we found that peptide D2A failed to stimulate migration of HEK-293 cells (that have no uPAR), while D2A-Ala succeeded in inhibiting VN-induced chemotaxis in the same cells. Thus, the induction of cell migration by D2A requires the presence of uPAR on the cell surface whereas the inhibition of VN chemotaxis just requires the binding of D2A-Ala to the integrin. These data reveal subtle differences in the agonistic and antagonistic mechanisms of peptides D2A and D2A-Ala and suggests that binding of uPAR can modify the conformation of integrins.

Furthermore, the agonistic effects of D2A and the dominant-negative behaviour of D1HD3-uPAR taken together suggest that uPAR can exert both a positive and a negative regulation on integrins. These data also suggest that besides the D2A epitope, other binding sites, most probably located on domain 3 of uPAR, are required for the induction of cell migration. This idea is also supported by the fact that LB6-D1D2 cells were found to be less sensitive to low doses of VN (FIG. 1). The following step-wise model might explain the regulatory effects of uPAR on integrins (FIG. 9). For the positive regulation, induction of cell migration, we suggest a three-steps mechanism. uPAR can first “Anchor” (or contact) integrins through a still undefined epitope. This would allow the subsequent interaction of the D2A sequence with the integrin, reinforcing the interaction and allowing signaling. Then integrin ligands such as VN can bind to the “activated” integrin, although it is possible that VN binding to integrin may be a requisite for the uPAR-integrin interaction. In our model, the functions of the D2A region, integrin binding and signaling, can be dissociated, as the GEEG region is required for signaling but not for binding. Whilst not wishing to be bound by any theory, it may be that the conformation of uPAR is important in this interaction. We call the conformation of uPAR able to activate signaling d3d2, as the contact would require both domains D2 and D3.

For the mechanism of negative regulation, we have taken into account the observation that D1HD3-uPAR has a dominant-negative effect. When uPAR contacts the integrin only through binding site(s) located within domain 3 of uPAR (mutant D1HD3), locking of the integrin in a signaling-inactive state would ensue. In that case, uPAR would be in a different conformation that we called d3 (FIG. 9).

In summary, in this invention we have identified a new chemotactic sequence located in domain 2 of uPAR. Peptide D2A, the synthetic peptide bearing that sequence, binds to integrins and acts through these receptors activating integrin-dependent signaling pathways amongst other pathways mediating chemotaxis. The two glutamic acid residues in the GEEG epitope are crucial for signaling. In addition, we have also identified a form of UPAR, D1HD3 that behaves as a dominant-negative mutant. These data suggest that the positive or negative regulation of integrin function by uPAR would depend on different conformations which may depend on different kinds of interactions. The positive regulation would be achieved by binding to multiple sites, including D2A located on domain 2 and, by extrapolation from the D1HD3 results, on domain 3 of uPAR. The negative regulation would rather be obtained by interactions involving site(s) located only on domain 3. Finally, by introducing mutations into the D2A sequence, we have generated a powerful integrin inhibitor, D2A-Ala. This inhibitor is extremely interesting and might be effective against physiological and pathological processes such as angiogenesis, inflammation, cardiovascular diseases, infectious diseases and cancers.

### Variants Fragments and Derivatives

The present invention also relates to variants, derivatives and fragments of the modulators of the present invention. Preferably, the variant sequences etc. are at least as biologically active as the sequences presented herein.

As used herein “biologically active” refers to a sequence having a similar structural function (but not necessarily to the same degree), and/or similar regulatory function (but not necessarily to the same degree), and/or similar biochemical function (but not necessarily to the same degree) of the naturally occurring sequence.

Preferably such variants, derivative and fragments comprise the sequence GEEG in the case of agonists, and the sequence GAAG in the case of antagonists.

The compounds may be derivatives of uPAR. In one embodiment is or is derivable form domain 2 of uPAR, or is a fragment thereof. In another embodiment the agonist is or is derivable from uPAR lacking domain 2, or a fragment thereof.

The term “protein” includes single-chain polypeptide molecules as well as multiple-polypeptide complexes where individual constituent polypeptides are linked by covalent or non-covalent means. The term “polypeptide” includes peptides of two or more amino acids in length, typically having, or having more than 5, 6, 7, 8, 9, 10, 11, 12, 13, 14, 15, 16, 17, 18, 19, 20, 21, 22, 23, 24, 25, 26, 27, 28, 29, or 30 amino acids. As used herein the terms protein and polypeptide and peptide may be assumed to be synonymous, protein merely being used in a general sense to indicate a relatively longer amino acid sequence than that present in a polypeptide, and polypeptide merely being used in a general sense to indicate a relatively longer amino acid sequence than that present in a peptide. Generally for ease of reference only we will simply refer to the term polypeptide.

It will be understood that amino acid sequences for use in the invention are not limited to the particular sequences or fragments thereof or sequences obtained from a particular protein but also include homologous sequences obtained from any source, for example related viral/bacterial proteins, cellular homologues and synthetic peptides, as well as variants or derivatives thereof. It must also be envisaged that the amino acid motifs of the present invention can be inserted into a different proteins, at their N-terminus, carboxy terminus or in the body of the protein.

Thus, the present invention covers variants, homologues or derivatives of the amino acid sequences for use in the present invention, as well as variants, homologues or derivatives of the nucleotide sequence coding for the amino acid sequences used in the present invention.

The present invention covers both synthetic and sequences obtainable from nature.

In the context of the present invention, a homologous sequence is taken to include an amino acid sequence which is at least 60, 70, 80 or 90% identical, preferably at least 95 or 98% identical at the amino acid level. In particular, homology should typically be considered with respect to those regions of the sequence known to be essential for chemotactic modulation, i.e. the GEEG and GAAG motifs, rather than non-essential neighbouring sequences. Homology can also be considered in terms of similarity (i.e. amino acid residues having similar chemical properties/functions), and in the context of the present invention it is preferred to express homology in terms of both sequence identity sequence similarity.

Homology comparisons can be conducted by eye, or more usually, with the aid of readily available sequence comparison programs. These commercially available computer programs can calculate % homology between two or more sequences.

% homology may be calculated over contiguous sequences, i.e. one sequence is aligned with the other sequence and each amino acid in one sequence directly compared with the corresponding amino acid in the other sequence, one residue at a time. This is called an “ungapped” alignment. Typically, such ungapped alignments are performed only over a relatively short number of residues (for example less than 50 contiguous amino acids).

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package (see below) the default gap penalty for amino acid sequences is −12 for a gap and −4 for each extension.

Calculation of maximum % homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al., 1984, Nucleic Acids Research 12:387). Examples of other software than can perform sequence comparisons include, but are not limited to, the BLAST package (see Ausubel et al., 1999 ibid7—Chapter 18), FASTA (Atschul et al., 1990, J. Mol. Biol., 403-410) and the GENEWORKS suite of comparison tools. Both BLAST and FASTA are available for offline and online searching (see Ausubel et al., 1999 ibid, pages 7-58 to 7-60). However it is preferred to use the GCG Bestfit program.

Although the final % homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). It is preferred to use the public default values for the GCG package, or in the case of other software, the default matrix, such as BLOSUM62.

Once the software has produced an optimal alignment, it is possible to calculate % homology, preferably % sequence identity. The software typically does this as part of the sequence comparison and generates a numerical result.

The terms “variant” or “derivative” in relation to the amino acid sequences of the present invention includes any substitution of, variation of, modification of, replacement of, deletion of or addition of one (or more) amino acids from or to the sequence providing the resultant amino acid sequence has the ability to modulate cell adhesion, migration, proliferation and/or differentiation activity.

Sequences may be modified for use in the present invention. Typically, modifications are made that maintain the activity of the sequence. Amino acid substitutions may be made, for example from 1, 2 or 3 to 10, 20 or 30 substitutions provided that the modified sequence retains the cell adhesion, migration, proliferation and/or differentiation modulation activity. Amino acid substitutions may include the use of non-naturally occurring analogues, for example to increase blood plasma half-life of a therapeutically administered polypeptide.

Conservative substitutions may be made, for example according to the Table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

Polypeptides of the invention also include fragments of the above mentioned polypeptides and variants thereof, including fragments of the sequences. Preferred fragments include those which include an epitope or binding domain. Suitable fragments will be at least about 5, e.g. 10, 12, 15 or 20 amino acids in length. They may also be less than 200, 100 or 50 amino acids in length. Polypeptide fragments of the proteins and allelic and species variants thereof may contain one or more (e.g. 2, 3, 5, or 10) substitutions, deletions or insertions, including conserved substitutions. Where substitutions, deletion and/or insertions have been made, for example by means of recombinant technology, preferably less than 20%, 10% or 5% of the amino acid residues depicted in the sequence listings are altered.

Proteins for use in the invention are typically made by recombinant means, for example as described below. However they may also be made by synthetic means using techniques well known to skilled persons such as solid phase synthesis. Various techniques for chemical synthesising peptides are reviewed by Borgia and Fields, 2000, TibTech 18: 243-251 and described in detail in the references contained therein.

Proteins for use in the invention may also be produced as fusion proteins, for example to aid in extraction and purification. Examples of fusion protein partners include glutathione-5-transferase (GST), 6×His, GAL4 (DNA binding and/or transcriptional activation domains) and β-galactosidase. It may also be convenient to include a proteolytic cleavage site between the fusion protein partner and the protein sequence of interest to allow removal of fusion protein sequences. Preferably the fusion protein will not hinder the activity of the protein of interest.

Proteins for use in the invention may be in a substantially isolated form. It will be understood that the protein may be mixed with carriers or diluents which will not interfere with the intended purpose of the protein and still be regarded as substantially isolated. A protein of the invention may also be in a substantially purified form, in which case it will generally comprise the protein in a preparation in which more than 90%, e.g. 95%, 98% or 99% of the protein in the preparation is a protein of the invention.

### Polynucleotides

Polynucleotides for use in the invention comprise nucleic acid sequences encoding the chemotactic modulators, including derivatives, variants, fragments etc. It will be understood by a skilled person that numerous different polynucleotides can encode the same protein as a result of the degeneracy of the genetic code. In addition, it is to be understood that skilled persons may, using routine techniques, make nucleotide substitutions that do not affect the protein sequence encoded by the polynucleotides of the invention to reflect the codon usage of any particular host organism in which the proteins for use in the invention are to be expressed.

Polynucleotides for use in the invention may comprise DNA or RNA. They may be single-stranded or double-stranded. They may also be polynucleotides which include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of the present invention, it is to be understood that the polynucleotides described herein may be modified by any method available in the art. Such modifications may be carried out in order to enhance the in vivo activity or life span of polynucleotides for use in the invention.

The terms “variant”, “homologue” or “derivative” in relation to the nucleotide sequence include any substitution of, variation of, modification of, replacement of, deletion of or addition of one (or more) nucleic acid from or to the sequence providing the resultant nucleotide sequence codes for a polypeptide having the capability to modify chemotactic activity.

As indicated above, with respect to sequence homology, preferably there is at least 75%, more preferably at least 85%, more preferably at least 90% homology to the sequences shown in the sequence listing herein. More preferably there is at least 95%, more preferably at least 98%, homology. Nucleotide homology comparisons may be conducted as described above. A preferred sequence comparison program is the GCG Wisconsin Bestfit program described above. The default scoring matrix has a match value of 10 for each identical nucleotide and −9 for each mismatch. The default gap creation penalty is −50 and the default gap extension penalty is −3 for each nucleotide.

The present invention also encompasses nucleotide sequences that are capable of hybridising selectively to the sequences presented herein, or any variant, fragment or derivative thereof, or to the complement of any of the above. Nucleotide sequences are preferably at least 15 nucleotides in length, more preferably at least 20, 30, 40 or 50 nucleotides in length.

The term “hybridization” as used herein shall include “the process by which a strand of nucleic acid joins with a complementary strand through base pairing” as well as the process of amplification as carried out in polymerase chain reaction technologies.

Polynucleotides for use in the invention capable of selectively hybridising to the nucleotide sequences presented herein, or to their complement, will be generally at least 70%, preferably at least 80 or 90% and more preferably at least 95% or 98% homologous to the corresponding nucleotide sequences presented herein over a region of at least 20, preferably at least 25 or 30, for instance at least 40, 60 or 100 or more contiguous nucleotides. Preferred polynucleotides for use in the invention will comprise regions homologous to the motifs, preferably at least 80 or 90% and more preferably at least 95% homologous to the motifs.

The term “selectively hybridizable” means that the polynucleotide used as a probe is used under conditions where a target polynucleotide for use in the invention is found to hybridize to the probe at a level significantly above background. The background hybridization may occur because of other polynucleotides present, for example, in the cDNA or genomic DNA library being screening. In this event, background implies a level of signal generated by interaction between the probe and a non-specific DNA member of the library which is less than 10 fold, preferably less than 100 fold as intense as the specific interaction observed with the target DNA. The intensity of interaction may be measured, for example, by radiolabelling the probe, e.g. with ³²P..

Hybridization conditions are based on the melting temperature (Tm) of the nucleic acid binding complex, as taught in Berger and Kimmel (1987, Guide to Molecular Cloning Techniques, Methods in Enzymology, Vol 152, Academic Press, San Diego Calif.), and confer a defined “stringency” as explained below.

Maximum stringency typically occurs at about Tm-5° C. (5° C. below the Tm of the probe); high stringency at about 5° C. to 10° C. below Tm; intermediate stringency at about 10° C. to 20° C. below Tm; and low stringency at about 20° C. to 25° C. below Tm. As will be understood by those of skill in the art, a maximum stringency hybridization can be used to identify or detect identical polynucleotide sequences while an intermediate (or low) stringency hybridization can be used to identify or detect similar or related polynucleotide sequences.

In a preferred aspect, the present invention covers nucleotide sequences that can hybridise to the nucleotide sequence of the present invention under stringent conditions (e.g. 65° C. and 0.1×SSC {1×SSC=0.15 M NaCl, 0.015 M Na₃ Citrate pH 7.0).

Where the polynucleotide for use in the invention is double-stranded, both strands of the duplex, either individually or in combination, are encompassed by the present invention. Where the polynucleotide is single-stranded, it is to be understood that the complementary sequence of that polynucleotide is also included within the scope of the present invention.

Polynucleotides which are not 100% homologous to the sequences used in the present invention but fall within the scope of the invention can be obtained in a number of ways. Other variants of the sequences described herein may be obtained for example by probing DNA libraries made from a range of individuals, for example individuals from different populations. In addition, other viral/bacterial, or cellular homologues particularly cellular homologues found in mammalian cells (e.g. rat, mouse, bovine and primate cells), may be obtained and such homologues and fragments thereof in general will be capable of selectively hybridising to the sequences shown in the sequence listing herein. Such sequences may be obtained by probing cDNA libraries made from or genomic DNA libraries from other animal species, and probing such libraries with probes comprising all or part of the human uPAR sequence under conditions of medium to high stringency. Similar considerations apply to obtaining species homologues and allelic variants of the protein or nucleotide sequences for use in the invention.

Variants and strain/species homologues may also be obtained using degenerate PCR which will use primers designed to target sequences within the variants and homologues encoding conserved amino acid sequences within the sequences of the present invention. Conserved sequences can be predicted, for example, by aligning the amino acid sequences from several variants/homologues. Sequence alignments can be performed using computer software known in the art. For example the GCG Wisconsin PileUp program is widely used.

The primers used in degenerate PCR will contain one or more degenerate positions and will be used at stringency conditions lower than those used for cloning sequences with single sequence primers against known sequences.

Alternatively, such polynucleotides may be obtained by site directed mutagenesis. This may be useful where for example silent codon changes are required to sequences to optimise codon preferences for a particular host cell in which the polynucleotide sequences are being expressed. Other sequence changes may be desired in order to introduce restriction enzyme recognition sites.

Polynucleotides of the invention may be used to produce a primer, e.g. a PCR primer, a primer for an alternative amplification reaction, a probe e.g. labelled with a revealing label by conventional means using radioactive or non-radioactive labels, or the polynucleotides may be cloned into vectors. Such primers, probes and other fragments will be at least 15, preferably at least 20, for example at least 25, 30 or 40 nucleotides in length, and are also encompassed by the term polynucleotides of the invention as used herein.

Polynucleotides such as a DNA polynucleotides and probes for use in the invention may be produced recombinantly, synthetically, or by any means available to those of skill in the art. They may also be cloned by standard techniques.

In general, primers will be produced by synthetic means, involving a step wise manufacture of the desired nucleic acid sequence one nucleotide at a time. Techniques for accomplishing this using automated techniques are readily available in the art.

Longer polynucleotides will generally be produced using recombinant means, for example using a PCR (polymerase chain reaction) cloning techniques. This will involve making a pair of primers (e.g. of about 15 to 30 nucleotides) flanking a region of the lipid targeting sequence which it is desired to clone, bringing the primers into contact with mRNA or cDNA obtained from an animal or human cell, performing a polymerase chain reaction under conditions which bring about amplification of the desired region, isolating the amplified fragment (e.g. by purifying the reaction mixture on an agarose gel) and recovering the amplified DNA. The primers may be designed to contain suitable restriction enzyme recognition sites so that the amplified DNA can be cloned into a suitable cloning vector

### Nucleotide Vectors

Polynucleotides of the invention can be incorporated into a recombinant replicable vector. The vector may be used to replicate the nucleic acid in a compatible host cell. Thus in a further embodiment, the invention provides a method of making polynucleotides for use in the invention by introducing a polynucleotide of the invention into a replicable vector, introducing the vector into a compatible host cell, and growing the host cell under conditions which bring about replication of the vector. The vector may be recovered from the host cell. Suitable host cells include bacteria such as E. coli, yeast, mammalian cell lines and other eukaryotic cell lines, for example insect Sf9 cells.

Preferably, a polynucleotide of the invention in a vector is operably linked to a control sequence that is capable of providing for the expression of the coding sequence by the host cell, i.e. the vector is an expression vector. The term “operably linked” means that the components described are in a relationship permitting them to function in their intended manner. A regulatory sequence “operably linked” to a coding sequence is ligated in such a way that expression of the coding sequence is achieved under condition compatible with the control sequences.

The control sequences may be modified, for example by the addition of further transcriptional regulatory elements to make the level of transcription directed by the control sequences more responsive to transcriptional modulators.

Vectors of the invention may be transformed or transfected into a suitable host cell as described below to provide for expression of a protein of the invention. This process may comprise culturing a host cell transformed with an expression vector as described above under conditions to provide for expression by the vector of a coding sequence encoding the protein, and optionally recovering the expressed protein.

The vectors may be for example, plasmid or virus vectors provided with an origin of replication, optionally a promoter for the expression of the said polynucleotide and optionally a regulator of the promoter. The vectors may contain one or more selectable marker genes, for example an ampicillin resistance gene in the case of a bacterial plasmid or a neomycin resistance gene for a mammalian vector. Vectors may be used, for example, to transfect or transform a host cell.

Vectors/polynucleotides for use in the invention may be introduced into suitable host cells using a variety of techniques known in the art, such as transfection, transformation and electroporation. Where vectors/polynucleotides of the invention are to be administered to animals, several techniques are known in the art, for example infection with recombinant viral vectors such as retroviruses, herpes simplex viruses and adenoviruses, direct injection of nucleic acids and biolistic transformation.

### Protein Expression and Purification

Host cells comprising polynucleotides of the invention may be used to express proteins for use in the invention. Host cells may be cultured under suitable conditions which allow expression of the proteins of the invention. Expression of the proteins of the invention may be constitutive such that they are continually produced, or inducible, requiring a stimulus to initiate expression. In the case of inducible expression, protein production can be initiated when required by, for example, addition of an inducer substance to the culture medium, for example dexamethasone or IPTG.

Proteins for use in the invention can be extracted from host cells by a variety of techniques known in the art, including enzymatic, chemical and/or osmotic lysis and physical disruption.

### Assays

The present invention also provides a method of screening compounds to identify agonists and antagonists to cell adhesion, migration, proliferation and/or differentiation. Candidate compounds may be identified from a variety of sources, for example, cells, cell-free preparations, chemical libraries, peptide and gene libraries, and natural product mixtures. Such agonists or antagonists or inhibitors so-identified may be natural or modified substrates, ligands, receptors, enzymes, etc., as the case may be, of for example uPAR; or may be structural or functional mimetics thereof (see Coligan et al., Current Protocols in Immunology 1(2):Chapter 5 (1991)).

The screening method may simply measure the binding of a candidate compound to the epitope GEEG or its modified form GAAG by means of a label directly or indirectly associated with the candidate compound. Alternatively, the screening method may involve competition with a labeled competitor. Further, these screening methods may test whether the candidate compound results in a signal generated by activation or inhibition of cell adhesion, migration, proliferation and/or differentiation, using detection systems appropriate to the cells bearing the receptor. A compound which binds but does not elicit a response identifies that compound as an antagonist. An antagonist compound is also one which binds and produces an opposite response.

One assay contemplated by the invention is a two-hybrid screen. The two-hybrid system was developed in yeast [Chien et al., Proc. Natl. Acad. Sci. USA, 88: 9578-9582 (1991)] and is based on functional in vivo reconstitution of a transcription factor which activates a reporter gene. Other assays for identifying proteins that interact with the uPAR epitope or its modified form may involve immobilizing uPAR or a test protein, detectably labelling the nonimmobilized binding partner, incubating the binding partners together and determining the amount of label bound. Bound label indicates that the test protein interacts with the epitope or its modified form.

Another type of assay for identifying cell adhesion, migration, proliferation and/or differentiation modulating proteins involves immobilizing uPAR or a fragment thereof containing the epitope or its modified form on a solid support coated (or impregnated with) a fluorescent agent, labelling a test protein with a compound capable of exciting the fluorescent agent, contacting the immobilized uPAR with the labelled test protein, detecting light emission by the fluorescent agent, and identifying interacting proteins as test proteins which result in the emission of light by the fluorescent agent. Alternatively, the putative interacting protein may be immobilized and uPAR may be labelled in the assay.

Also comprehended by the present invention are antibody products (e.g., monoclonal and polyclonal antibodies, single chain antibodies, chimeric antibodies, CDR-grafted antibodies and antigen-binding fragments thereof) and other binding proteins (such as those identified in the assays above) which are specific for the motifs of the invention, or modified forms thereof. Binding proteins can be developed using isolated natural or recombinant enzymes. The binding proteins are useful, in turn, for purifying recombinant and naturally occurring enzymes and identifying cells producing such enzymes. Assays for the detection and quantification of proteins in cells and in fluids may involve a single antibody substance or multiple antibody substances in a “sandwich” assay format to determine cytological analysis of uPAR protein levels. Anti-idiotypic antibodies are also contemplated.

Delivery of a gene coding for a protein that mimics functional uPAR to appropriate cells may be effected in vivo or ex vivo by use of viral vectors (e.g., adenovirus, adeno-associated virus, or a retrovirus) or ex vivo by use of physical DNA transfer methods (e.g., liposomes or chemical treatments). For reviews of gene therapy technology see Friedmann, Science, 244: 1275-1281 (1989); Verma, Scientific American: 68-84 (1990); and Miller, Nature, 357: 455-460 (1992). Alternatively, it is contemplated that in other human disease states preventing the expression of or inhibiting cell adhesion, migration, proliferation and/or differentiation will be useful in treating the disease states. It is contemplated that antisense therapy or gene therapy could be applied to negatively regulate chemotaxis. Antisense nucleic acids (preferably 10 to 20 base pair oligonucleotides) capable of specifically binding to the motifs or modified forms thereof are introduced into cells (e.g., by a viral vector or colloidal dispersion system such as a liposome). The antisense nucleic acid binds to the target sequence in the cell and prevents transcription or translation of the target sequence. Phosphothioate and methylphosphate antisense oligonucleotides are specifically contemplated for therapeutic use by the invention. The antisense oligonucleotides may be further modified by poly-L-lysine, transferrin polylysine, or cholesterol moieties at their 5′ end.

Small molecule-based therapies are particularly preferred because such molecules are more readily absorbed after oral administration and/or have fewer potential antigenic determinants than larger, protein-based pharmaceuticals. In light of the present disclosure, one of ordinary skill in the art will be able to develop drug screening methodologies which will be useful in the identification of candidate small molecule pharmaceuticals for the treatment of diseases. In particular, the skilled person will be able to screen large libraries of small molecules in order to identify those which bind to the normal and/or mutant/acetylated peptides containing the motifs and which, therefore, are candidates for modifying the in vivo activity of the normal or mutant/acetylated peptides. Furthermore, the skilled person will be able to identify small molecules which selectively or preferentially bind to the motifs or their modified forms.

Methods for screening small molecule libraries for candidate protein-binding molecules are well known in the art and, in light of the present disclosure, may now be employed to identify compounds which bind to the normal or mutant forms of the motifs of the present invention.

As will be obvious to one of ordinary skill in the art, there are numerous other methods of screening individual small molecules or large libraries of small molecules (e.g., phage display libraries) to identify compounds which bind to normal or mutant motifs of the present invention. All of these methods comprise the step of mixing normal or mutant motifs with test compounds, allowing for binding (if any), and assaying for bound complexes.

Compounds which bind to normal or mutant or both forms of the motifs of the present invention may have utility in treatments.

Once identified by the methods described above, the candidate compounds may then be produced in quantities sufficient for pharmaceutical administration or testing or may serve as “lead compounds” in the design and development of new pharmaceuticals. For example, as is well known in the art, sequential modification of small molecules (e.g., amino acid residue replacement with peptides; functional group replacement with peptide or non-peptide compounds) is a standard approach in the pharmaceutical industry for the development of new pharmaceuticals. Such development generally proceeds from a “lead compound” which is shown to have at least some of the activity of the desired pharmaceutical. In particular, when one or more compounds having at least some activity of interest are identified, structural comparison of the molecules can greatly inform the skilled practitioner by suggesting portions of the lead compounds which should be conserved and portions which may be varied in the design of new candidate compounds. Thus, the present invention also provides a means of identifying lead compounds which may be sequentially modified to produce new candidate compounds for use in the treatment of diseases. These new compounds then may be tested both for binding (e.g., in the binding assays described above) and for therapeutic efficacy (e.g., in the animal models described herein). This procedure may be iterated until compounds having the desired therapeutic activity and/or efficacy are identified.

Compounds identified by this method will have potential utility in modifying cell adhesion, migration, proliferation and/or differentiation in vivo. These compounds may be further tested in the animal models disclosed and enabled herein to identify those compounds having the most potent in vivo effects. In addition, as described above with respect to small molecules having cell adhesion, migration, proliferation and/or differentiation modulating activity, these molecules may serve as “lead compounds” for the further development of pharmaceuticals by, for example, subjecting the compounds to sequential modifications, molecular modelling, and other routine procedures employed in rational drug design.

The invention, thus, provides methods which may be used to identify compounds which may act, for example, as regulators or modulators such as agonists and antagonists, partial agonists, inverse agonists, activators, co-activators, and inhibitors. Accordingly, the invention provides reagents and methods for regulating the expression of a polynucleotide or a polypeptide associated with cell adhesion, migration, proliferation and/or differentiation. Reagents that modulate the expression, stability, or amount of a polynucleotide or the activity of the polypeptide may be a protein, a peptide, a peptidomimetic, a nucleic acid, a nucleic acid analogue (e.g., peptide nucleic acid, locked nucleic acid), or a small molecule.

One aspect of the present invention involves methods for screening test compounds to identify agents that inhibit urokinase-type plasminogen activator (uPA) formation or activity, uPA receptor (UPAR) formation or ligand binding or activity. Another aspect of the invention involves active pharmaceutical agents that inhibit one or more drug targets such as uPA and uPAR. Yet another aspect involves pharmaceutical agents that are active in treating or preventing diseases or conditions associated with cell adhesion, migration, proliferation and/or differentiation.

The invention also provides a method of screening to identify an agent useful for treating or preventing diseases or conditions associated with cell adhesion, migration, proliferation and/or differentiation, which comprises (i) providing a pool of test agents; (ii) determining whether any test agent from the pool inhibits the activity of at least one member selected from the group consisting of urokinase-type plasminogen activator and urokinase-type plasminogen activator receptor, and (iii) selecting any test agent from the pool that inhibits the activity of at least one member as an agent useful for treating or preventing diseases or conditions associated with cell adhesion, migration, proliferation and/or differentiation. The method may optionally comprise a step of selecting the pool of test agents prior to step (i).

In a one embodiment, the determining step comprises (a) contacting a test agent from the pool with urokinase-type plasminogen activator and a substrate to form a product; (b) measuring the level of the substrate or the product after the contacting step; (c) comparing the substrate level to a substrate control value or the product level to a product control value; and (d) selecting any test agent for which the substrate level is higher than the substrate control value or for which the product level is lower than the product control value as an agent that inhibits the urokinase-type plasminogen activator.

Optionally, the substrate is L-pyroglutamyl-glycyl-L-arginine-p-nitroaniline hydrochloride and the product p-nitroaniline dihydrochloride.

In another embodiment, the determining step comprises (a) contacting the test agent with urokinase-type plasminogen activator receptor and urokinase-type plasminogen activator; (b) measuring the level of binding between the urokinase-type plasminogen activator receptor and urokinase-type plasminogen activator substrate after the contacting step; (c) comparing the level of binding to a control value; and (c) selecting any test agent for which the level of binding is lower than the control value as an agent useful for treating or preventing diseases or conditions associated with cell adhesion, migration, proliferation and/or differentiation.

In another embodiment, the test agent comprises an antigen-binding fragment of an antibody directed against the at least one of the motifs of the present invention.

The invention also provides for a method of identifying an agent as useful in treating diseases or conditions associated with cell adhesion, migration, proliferation and/or differentiation, which comprises administering a test agent to a mammal and determining whether the test agent inhibits urokinase-type plasminogen activator or urokinase-type plasminogen activator receptor in said mammal.

### Therapeutic Peptides

Peptides of the present invention may be administered therapeutically to patients. It is preferred to use proteins that do not consist solely of naturally-occurring amino acids but which have been modified, for example to reduce immunogenicity, to increase circulatory half-life in the body of the patient, to enhance bioavailability and/or to enhance efficacy and/or specificity.

A number of approaches have been used to modify proteins for therapeutic application. One approach is to link the peptides or proteins to a variety of polymers, such as polyethylene glycol (PEG) and polypropylene glycol (PPG)—see for example U.S. Pat. Nos. 5,091,176, 5,214,131 and U.S. Pat. No. 5,264,209.

Replacement of naturally-occurring amino acids with a variety of uncoded or modified amino acids such as D-amino acids and N-methyl amino acids may also be used to modify proteins

Another approach is to use bifunctional crosslinkers, such as N-succinimidyl 3-(2 pyridyldithio) propionate, succinimidyl 6-[3-(2 pyridyldithio) propionamido] hexanoate, and sulfosuccinimidyl 6-[3-(2 pyridyldithio) propionamido]hexanoate (see U.S. Pat. No. 5,580,853).

It may be desirable to use derivatives of the proteins of the invention which are conformationally constrained. Conformational constraint refers to the stability and preferred conformation of the three-dimensional shape assumed by a protein. Conformational constraints include local constraints, involving restricting the conformational mobility of a single residue in a protein; regional constraints, involving restricting the conformational mobility of a group of residues, which residues may form some secondary structural unit; and global constraints, involving the entire protein structure.

The active conformation of the protein may be stabilised by a covalent modification, such as cyclization or by incorporation of gamma-lactam or other types of bridges. For example, side chains can be cyclized to the backbone so as create a L-gamma-lactam moiety on each side of the interaction site. See, generally, Hruby et al., “Applications of Synthetic Peptides,” in Synthetic Peptides: A User's Guide: 259-345 (W. H. Freeman & Co. 1992). Cyclization also can be achieved, for example, by formation of cysteine bridges, coupling of amino and carboxy terminal groups of respective terminal amino acids, or coupling of the amino group of a Lys residue or a related homolog with a carboxy group of Asp, Glu or a related homolog. Coupling of the alpha-amino group of a polypeptide with the epsilon-amino group of a lysine residue, using iodoacetic anhydride, can be also undertaken. See Wood and Wetzel, 1992, Int'l J. Peptide Protein Res. 39: 533-39.

Another approach described in U.S. Pat. No. 5,891,418 is to include a metal-ion complexing backbone in the protein structure. Typically, the preferred metal-peptide backbone is based on the requisite number of particular coordinating groups required by the coordination sphere of a given complexing metal ion. In general, most of the metal ions that may prove useful have a coordination number of four to six. The nature of the coordinating groups in the protein chain includes nitrogen atoms with amine, amide, imidazole, or guanidino functionalities; sulfur atoms of thiols or disulfides; and oxygen atoms of hydroxy, phenolic, carbonyl, or carboxyl functionalities. In addition, the protein chain or individual amino acids can be chemically altered to include a coordinating group, such as for example oxime, hydrazino, sulfhydryl, phosphate, cyano, pyridino, piperidino, or morpholino. The protein construct can be either linear or cyclic, however a linear construct is typically preferred. One example of a small linear peptide is Gly-Gly-Gly-Gly which has four nitrogens (an N₄ complexation system) in the back bone that can complex to a metal ion with a coordination number of four.

A further technique for improving the properties of therapeutic proteins is to use non-peptide peptidomimetics. A wide variety of useful techniques may be used to elucidating the precise structure of a protein. These techniques include amino acid sequencing, x-ray crystallography, mass spectroscopy, nuclear magnetic resonance spectroscopy, computer-assisted molecular modelling, peptide mapping, and combinations thereof. Structural analysis of a protein generally provides a large body of data which comprise the amino acid sequence of the protein as well as the three-dimensional positioning of its atomic components. From this information, non-peptide peptidomimetics may be designed that have the required chemical functionalities for therapeutic activity but are more stable, for example less susceptible to biological degradation. An example of this approach is provided in U.S. Pat. No. 5,811,512.

Techniques for chemically synthesising therapeutic proteins of the invention are described in the above references and also reviewed by Borgia and Fields, 2000, TibTech 18: 243-251 and described in detail in the references contained therein.

### Antibodies

The invention also provides monoclonal or polyclonal antibodies to polypeptides of the invention or fragments thereof. Thus, the present invention further provides a process for the production of monoclonal or polyclonal antibodies to polypeptides of the invention. As indicated above such antibodies may be useful in treatment or diagnosis or in any of the assays described herein.

If polyclonal antibodies are desired, a selected mammal (e.g., mouse, rabbit, goat, horse, etc.) is immunised with an immunogenic polypeptide bearing a GEEG or GAAG epitope(s). Serum from the immunised animal is collected and treated according to known procedures. If serum containing polyclonal antibodies to a GAAG or GEEG epitope contains antibodies to other antigens, the polyclonal antibodies can be purified by immunoaffinity chromatography. Techniques for producing and processing polyclonal antisera are known in the art. In order that such antibodies may be made, the invention also provides polypeptides of the invention or fragments thereof haptenised to another polypeptide for use as immunogens in animals or humans.

Monoclonal antibodies directed against GAAG or GEEG epitopes in the polypeptides of the invention can also be readily produced by one skilled in the art. The general methodology for making monoclonal antibodies by hybridomas is well known. Immortal antibody-producing cell lines can be created by cell fusion, and also by other techniques such as direct transformation of B lymphocytes with oncogenic DNA, or transfection with Epstein-Barr virus. Panels of monoclonal antibodies produced against GAAG or GEEG epitopes can be screened for various properties; i.e., for isotype and epitope affinity.

An alternative technique involves screening phage display libraries where, for example the phage express scFv fragments on the surface of their coat with a large variety of complementarity determining regions (CDRs). This technique is well known in the art.

Antibodies, both monoclonal and polyclonal, which are directed against GAAG or GEEG epitopes are particularly useful in diagnosis, and those which are neutralising are useful in passive immunotherapy. Monoclonal antibodies, in particular, may be used to raise anti-idiotype antibodies. Anti-idiotype antibodies are immunoglobulins which carry an “internal image” of the antigen of the agent against which protection is desired.

Techniques for raising anti-idiotype antibodies are known in the art. These anti-idiotype antibodies may also be useful in therapy.

For the purposes of this invention, the term “antibody”, unless specified to the contrary, includes fragments of whole antibodies which retain their binding activity for a target antigen. Such fragments include Fv, F(ab′) and F(ab′)₂ fragments, as well as single chain antibodies (scFv). Furthermore, the antibodies and fragments thereof may be humanised antibodies, for example as described in EP-A-239400.

### Therapeutic Uses

This includes any therapeutic application that can benefit a human or non-human animal. The treatment of mammals is particularly preferred. Both human and veterinary treatments are within the scope of the present invention.

Treatment may be in respect of an existing condition or it may be prophylactic. It may be of an adult, a juvenile, an infant, a foetus, or a part of any of the aforesaid (e.g. an organ, tissue, cell, or nucleic acid molecule).

uPAR is understood to coordinate signalling for cell adhesion, migration and growth, thus influencing cellular behaviour in angiogenesis, inflammation, wound repair, infectious diseases and tumor progression.

For example, uPA is synthesized and secreted by tumor cells. In solution or upon binding to its receptor uPAR; uPA generates plasmin, which in turn degrades extracellular matrix components leading to invasion and metastasis. It will therefore be appreciated that inhibition of the uPA/uPAR interaction using the inhibitors of the present invention could lead to reduction of invasion and spread of tumor cells.

In one embodiment the peptides, antibodies, compounds or pharmaceutical compositions etc., (also referred to herein as “compounds”) may be used to treat or prevent cancer including but not limited to cancer of the lung, pancreas, breast, colon, larynx, kidney, uterus, prostate, bladder or ovary. In one embodiment, the cancer comprises a solid tumor. The present invention is also useful in relation to adenocarcinomas, such as small cell lung cancer. The present invention may be useful in relation to other types of cancer, such as leukaemia.

The process by which new blood capillaries grow into a wound space after injury is known as angiogenesis. Angiogenesis also occurs in many other situations, including solid tumor growth and metastasis, rheumatoid arthritis, psoriasis, scleroderma, capillary proliferation in atherosclerotic plaques and osteoporosis; and the three common causes of blindness—diabetic retinopathy, retrolental fibroplasia and neovascular glaucoma—in fact, diseases of the eye are almost always accompanied by vascularization. The process of wound angiogenesis has many features in common with tumor angiogenesis. It will be appreciated that the compounds of the present invention may be used to control angiogenesis, and in particular the inhibitors of the present invention may be useful in inhibiting angiogenesis and thus in treating diseases associated with angiogenesis.

The arthritis can be caused by a degenerative joint disease, such as for example, rheumatoid arthritis, psoriatic arthritis, infectious arthritis, juvenile rheumatoid arthritis; osteoarthritis, or spondyloarthropaties. In a preferred embodiment, the arthritis is rheumatoid arthritis, and the mammal is a human.

The present invention may also be useful in treating vascular conditions associated with chronic inflammatory processes, such as atherosclerosis and restenosis. In particular, the compounds of the present invention may be useful in the treatment of such diseases. The present invention may also be useful in treating other disorders associated with inflammation.

Thus, in another embodiment, the compounds may be used to treat or prevent diseases involving angiogenisis, such as diseases associated with αvβ3 expression.

As discussed above, angiogenesis is a process of tissue vascularization that involves the growth of new developing blood vessels into a tissue, and is also referred to as neo-vascularization. The process is mediated by the infiltration of endothelial cells and smooth muscle cells. The process is believed to proceed in any one of three ways: the vessels can sprout from pre-existing vessels, de-novo development of vessels can arise from precursor cells (vasculogenesis), or existing small vessels can enlarge in diameter (Blood et al., 1990).

There are a variety of diseases in which angiogenesis is believed to be important, referred to as angiogenic diseases, including but not limited to, inflammatory disorders such as immune and non-immune inflammation, arthritis, disorders associated with inappropriate or inopportune invasion of vessels such as diabetic retinopathy, macular degeneration, neovascular glaucoma, restenosis, capillary proliferation in atherosclerotic plaques and osteoporosis, and cancer associated disorders, such as solid tumors, solid tumor metastases, angiofibromas, retrolental fibroplasia, hemangiomas, Kaposi sarcoma and the like which require neovascularization to support tumor growth.

Thus, methods which inhibit angiogenesis in a diseased tissue ameliorates symptoms of the disease and, depending upon the disease, can contribute to cure of the disease.

In another related embodiment, a tissue to be treated is a retinal tissue of a patient with diabetic retinopathy, macular degeneration or neovascular glaucoma and the angiogenesis to be inhibited is retinal tissue angiogenesis where there is neovascularization of retinal tissue.

In an additional related embodiment, a tissue to be treated is a tumor tissue of a patient with a solid tumor, a metastases, a skin cancer, a breast cancer, a hemangioma or angiofibroma and the like cancer, and the angiogenesis to be inhibited is tumor tissue angiogenesis where there is neovascularization of a tumor tissue. Typical solid tumor tissues treatable by the present methods include lung, pancreas, breast, colon, laryngeal, ovarian, and the like tissues.

Inhibition of tumor tissue angiogenesis is a particularly preferred embodiment because of the important role neovascularization plays in tumor growth. In the absence of neovascularization of tumor tissue, the tumor tissue does not obtain the required nutrients, slows in growth, ceases additional growth, regresses and ultimately becomes necrotic resulting in killing of the tumor.

The methods are also particularly effective against the formation of metastases because (1) their formation requires vascularization of a primary tumor so that the metastatic cancer cells can exit the primary tumor and (2) their establishment in a secondary site requires neovascularization to support growth of the metastases.

In a related embodiment, the invention contemplates the practice of the method in conjunction with other therapies such as conventional chemotherapy directed against solid tumors and for control of establishment of metastases. The administration of angiogenesis inhibitor is typically conducted during or after chemotherapy, although it is preferably to inhibit angiogenesis after a regimen of chemotherapy at times where the tumor tissue will be responding to the toxic assault by inducing angiogenesis to recover by the provision of a blood supply and nutrients to the tumor tissue. In addition, it is preferred to administer the angiogenesis inhibition methods after surgery where solid tumors have been removed as a prophylaxis against metastases.

Insofar as the present methods apply to inhibition of tumor neovascularization, the methods can also apply to inhibition of tumor tissue growth, to inhibition of tumor metastases formation, and to regression of established tumors.

Restenosis is a process of smooth muscle cell (SMC) migration and proliferation at the site of percutaneous transluminal coronary angioplasty which hampers the success of angioplasty. The migration and proliferation of SMC's during restenosis can be considered a process of angiogenesis which is inhibited by the present methods. Therefore, the invention also contemplates inhibition of restenosis by inhibiting angiogenesis according to the present methods in a patient following angioplasty procedures.

The compounds of the invention can be used in combined, separated or sequential preparations, also with other diagnostic or therapeutic substances.

The modulators of the present invention may also be useful in treating Graft-versus-Host disease.

Both bacterial and viral infections (like borrheliosis, AIDS) induce uPAR and uPA and the level of circulating soluble uPAR in HIV-positive patients is a very strong negative prognostic marker (Sidenius et al., 2000). Moreover, uPAR is induced by HIV infection of macrophage-type cells (Nykjaer et al., 1994). In particular, the inhibitors of the present invention may be useful in the treatment of such diseases.

The present invention may also be useful in treating infectious disorders due to other agents.

It is has also been proposed that uPAR deficiency accelerates fibrosis. Thus, it will be appreciated that the modulators of the present invention may be used to control diseases associated with fibrosis, and also may be used in the control of tissue remodelling and wound healing.

The compounds of the present invention and in particular the agonists of the present invention may be useful in the treatment of diseases in which stem cell stimulation and/or mobilization is desirable.

Stem cell transplantation can offer a chance for cure of a variety of disorders, particularly in the pediatric population. Malignant diseases treated with allogenic stem cell transplantation include Acute Lymphocytic Leukemia, Acute Myeloid Leukemia, Chronic Myelogenous Leukemia and Myelodysplastic Syndromes. Autologous stem cell transplantation is effective in treating solid tumors such as neuroblastoma, Wilm's tumor, lymphoma (Hodgkin's and non-Hodgkin's) and brain tumors.

Stem cells for use in transplantations may be harvested from the peripheral blood. Under normal conditions, hematopoietic stem cells and progenitor cells reside in the bone marrow compartment and are rarely found in circulation. In order to obtain sufficient numbers of these cells from the peripheral blood for clinical use, the stem cells and progenitor cells must be mobilized from the marrow compartment to the peripheral blood. Three methods are commonly used for this mobilization: a) myelosuppressive chemotherapy, b) administration of hematopoietic growth factors, or c) the combined use of a) and b). It will be appreciated that the activators of the present invention may be used in such a mobilization process, either on their own or in combination with other methods. This is especially true in view of data that demonstrates a correlation between uPAR and stem cell mobilization (Selleri et al., 2004), as well as the fact that the uPAR KO mice are deficient in hematopoietic stem cell mobilization. The present peptides may be used in particular to stimulate hematopoietic CD34-positive stem cells, such as KG-1 cells.

A compound of the present invention is administered to a patient, preferably a human, suffering from a disease characterized by cell migration, cell invasion or cell proliferation, angiogenesis or metastasis. Such diseases or conditions may include primary growth of solid tumors or leukemias and lymphomas, metastasis, invasion and/or growth of tumor metastases, benign hyperplasias, atherosclerosis, myocardial angiogenesis, angiofibroma, arteriovenous malformations, post-balloon angioplasty vascular restenosis, neointima formation following vascular trauma, vascular graft restenosis, coronary collateral formation, deep venous thrombosis, ischemic limb angiogenesis, telangiectasia, pyogenic granuloma, corneal diseases, rubeosis, neovascular glaucoma, diabetic and other retinopathy, retrolental fibroplasia, diabetic neovascularization, macular degeneration, endometriosis, arthritis, fibrosis associated with chronic inflammatory conditions including psoriasisscleroderma, hemangioma, hemophilic joints, hypertrophic scars, Osler-Weber syndrome, psoriasis, pyrogenic granuloma, retrolental fibroplasia, scleroderma, Von-Hippel-Landau syndrome, trachoma, vascular adhesions, lung fibrosis, chemotherapy-induced fibrosis, wound healing with scarring and fibrosis, peptic ulcers, fractures, keloids, and disorders of vasculogenesis, hematopoiesis, ovulation, menstruation, pregnancy and placentation, or any other disease or condition in which cell invasion or angiogenesis is pathogenic or undesired.

More recently, it has become apparent that angiogenesis inhibitors may play a role in preventing inflammatory angiogenesis and gliosis following traumatic spinal cord injury, thereby promoting the reestablishment of neuronal connectivity (Wamil etczl., Proc. Natl. Acad. Sci. 1998, 95: 13188-13193). Therefore, compounds are administered as soon as possible after traumatic spinal cord injury and for several days up to about two weeks thereafter to inhibit angiogenesis and gliosis that would sterically prevent reestablishment of neuronal connectivity. The treatment reduces the area of damage at the site of spinal cord injury and facilitates regeneration of neuronal function and thereby prevents paralysis. The compounds are expected also to protect axons from Wallerian degeneration, reverse aminobutyrate-mediated depolarization (occurring in traumatized neurons), and improve recovery of neuronal conductivity of isolated central nervous system cells and tissue in culture.

Further, in certain embodiments, compounds are administered to a patient, preferably a human, as a preventative measure against the above various diseases or disorders.

Thus, compounds may be administered as a preventative measure to a patient having a predisposition for a disease characterized by cell migration, cell invasion or cell proliferation, angiogenesis or metastasis. Accordingly, the compounds may be used for the prevention of one disease or disorder and concurrently treating another.

In one embodiment a compound is administered to a patient, preferably a human, in a diagnostically effective amount to detect or image a disease such as those listed above. Further, compounds may be used to detect or image diseases or conditions associated with undesired cell migration, invasion or proliferation such as those listed above by administering to a subject an diagnostically effective amount of compound. Antibodies may be diagnostically labeled and used, for example, to detect peptide-binding ligands or cellular binding sites/receptors (e.g., uPAR) either in the interior or on the surface of a cell. The disposition of the antibody during and after binding may be followed in vitro or in vivo by using an appropriate method to detect the label. Diagnostically labeled antibodies may be utilized in vivo for diagnosis and prognosis, for example, to image occult metastatic foci or for other types of in situ evaluations. For diagnostic applications, antibodies may include bound linker moieties, which are well known to those of skill in the art.

In situ detection of the labeled antibody may be accomplished by removing ahistological specimen from a subject and examining it by microscopy under appropriate conditions to detect the label. Those of ordinary skill will readily perceive that any of a wide variety of histological methods (such as staining procedures) can be modified in order to achievesuch in situ detection.

For diagnostic in vivo radioimaging, the type of detection instrument available is a major factor in selecting a radionuclide. The radionuclide chosen must have a type of decay which is detectable by a particular instrument. In general, any conventional method for visualizing diagnostic imaging can be utilized in accordance with this invention. Another factor in selecting a radionuclide for in vivo diagnosis is that its half-life be long enough so that the label is still detectable at the time of maximum uptake by the target tissue, but short enough so that deleterious irradiation of the host is minimized. In one preferred embodiment, a radionuclide used for in vivo imaging does not emit particles, but produces a large number of photons in a 140-200 keV range, which may be readily detected by conventional gamma cameras.

In vivo imaging may be used to detect occult metastases which are not observable by other methods. The expression of uPAR correlates with progression of diseases in cancer patients such that patients with late stage cancer have higher levels of uPAR in both their primary tumors and metastases. uPAR-targeted imaging could be used to stage tumors non-invasively or to detect other diseases which are associated with the presence of increased levels of UPAR (for example, restenosis that occurs following angioplasty).

The compounds may be used in diagnostic, prognostic or research procedures in conjunction with any appropriate cell, tissue, organ or biological sample of the desired animal species. By the term “biological sample” is intended any fluid or other material derived from the body of a normal or diseased subject, such as blood, serum, plasma, lymph, urine, saliva, tears, cerebrospinal fluid, milk, amniotic fluid, bile, ascites fluid, pus and the like. Also included within the meaning of this term is a organ or tissue extract and a culture fluid in which any cells or tissue preparation from the subject has been incubated.

Useful doses are defined as effective amount of the compound for the particular diagnostic measurement. Thus, an effective amount means an amount sufficient to be detected using the appropriate detection system e.g—, magnetic resonance imaging detector, gamma camera, etc. The minimum detectable amount will depend on the ratio of labeled antibody specifically bound to a tumor (signal) to the amount of labeled antibody either bound non-specifically or found free in plasma or in extracellular fluid.

The amount of the diagnostic composition to be administered depends on the precise antibody selected, the disease or condition, the route of administration, and the judgment of the skilled imaging professional. Generally, the amount of antibody needed for detectability in diagnostic use will vary depending on considerations such as age, condition, sex, and extent of disease in the patient, contraindications, if any, and other variables, and is to be adjusted by the individual physician or diagnostician.

### Administration

Compounds capable of affecting cell migration, cell adhesion, cell proliferation and/or cell differentiation for use in therapy are typically formulated for administration to patients with a pharmaceutically acceptable carrier or diluent to produce a pharmaceutical composition. The formulation will depend upon the nature of the compound identified and the route of administration but typically they can be formulated for topical, parenteral, intramuscular, intravenous, intra-peritoneal, intranasal inhalation, lung inhalation, intradermal or intra-articular administration. The compound may be used in an injectable form. It may therefore be mixed with any vehicle which is pharmaceutically acceptable for an injectable formulation, preferably for a direct injection at the site to be treated, although it may be administered systemically.

The pharmaceutically acceptable carrier or diluent may be, for example, sterile isotonic saline solutions, or other isotonic solutions such as phosphate-buffered saline. The compounds of the present invention may be admixed with any suitable binder(s), lubricant(s), suspending agent(s), coating agent(s), solubilising agent(s). It is also preferred to formulate the compound in an orally active form.

In general, a therapeutically effective daily oral or intravenous dose of the compounds of the invention is likely to range from 0.01 to 50 mg/kg body weight of the subject to be treated, preferably 0.1 to 20 mg/kg. The compounds may also be administered by intravenous infusion, at a dose which is likely to range from 0.001-10 mg/kg/hr.

Tablets or capsules of the compounds may be administered singly or two or more at a time, as appropriate. It is also possible to administer the compounds in sustained release formulations.

Alternatively, the compounds of the invention can be administered by inhalation or in the form of a suppository or pessary, or they may be applied topically in the form of a lotion, solution, cream, ointment or dusting powder. An alternative means of transdermal administration is by use of a skin patch. For example, they can be incorporated into a cream consisting of an aqueous emulsion of polyethylene glycols or liquid paraffin. They can also be incorporated, at a concentration of between 1 and 10% by weight, into an ointment consisting of a white wax or white soft paraffin base together with such stabilisers and preservatives as may be required.

For some applications, preferably the compositions are administered orally in the form of tablets containing excipients such as starch or lactose, or in capsules or ovules either alone or in admixture with excipients, or in the form of elixirs, solutions or suspensions containing flavouring or colouring agents.

The compositions (as well as the compounds alone) can also be injected parenterally, for example intracavernosally, intravenously, intramuscularly or subcutaneously. In this case, the compositions will comprise a suitable carrier or diluent.

For parenteral administration, the compositions are best used in the form of a sterile aqueous solution which may contain other substances, for example enough salts or monosaccharides to make the solution isotonic with blood.

For buccal or sublingual administration the compositions may be administered in the form of tablets or lozenges which can be formulated in a conventional manner.

For oral, parenteral, buccal and sublingual administration to subjects (such as patients), the daily dosage level of the compounds of the present invention and their pharmaceutically acceptable salts and solvates may typically be from 10 to 500 mg (in single or divided doses). Thus, and by way of example, tablets or capsules may contain from 5 to 100 mg of active compound for administration singly, or two or more at a time, as appropriate. As indicated above, the physician will determine the actual dosage which will be most suitable for an individual patient and it will vary with the age, weight and response of the particular patient. It is to be noted that whilst the above-mentioned dosages are exemplary of the average case there can, of course, be individual instances where higher or lower dosage ranges are merited and such dose ranges are within the scope of this invention.

The composition may be formulated such that administration daily, weekly or monthly will provide the desired daily dosage. It will be appreciated that the composition may be conveniently formulated for administrated less frequently, such as every 2, 4, 6, 8, 10 or 12 hours.

Polynucleotides/vectors encoding polypeptide components may be administered directly as a naked nucleic acid construct, preferably further comprising flanking sequences homologous to the host cell genome.

Uptake of naked nucleic acid constructs by mammalian cells is enhanced by several known transfection techniques for example those including the use of transfection agents. Example of these agents include cationic agents (for example calcium phosphate and DEAE-dextran) and lipofectants (for example Lipofectam™ and Transfectam™). Typically, nucleic acid constructs are mixed with the transfection agent to produce a composition.

Preferably the polynucleotide or vector of the invention is combined with a pharmaceutically acceptable carrier or diluent to produce a pharmaceutical composition. Suitable carriers and diluents include isotonic saline solutions, for example phosphate-buffered saline. The composition may be formulated for parenteral, intramuscular, intravenous, subcutaneous, intraocular or transdermal administration.

The routes of administration and dosages described are intended only as a guide since a skilled practitioner will be able to determine readily the optimum route of administration and dosage for any particular patient depending on, for example, the age, weight and condition of the patient.

Various preferred features and embodiments of the invention will now be described further with reference to the following non-limiting examples.

### EXAMPLES

### Materials and Cell Culture

Mouse LB6 and NIH 3T3 parental and transfected cells, human embryonic kidney cells HEK-293 and transfected HEK-293-uPAR, and rat smooth muscle cells (RSMC, Bayer Research Laboratories, Milano, Italy) were cultured in DMEM plus 10% FCS. LB6 cells express murine u-PAR but neither plasminogen activators as shown by RT-PCR) and zymographic analysis, nor PAI-1 (as shown by reverse zymography and RT-PCR) and have undetectable metalloprotease activity (Riittinen et al., 1996; Ossowski et al., 1991; Roldan et al., 1990). NIH 3T3 cells do not express u-PA or t-PA, except after phorbol ester induction and growth factor receptors activation, but they express low levels of PAI-1 (reverse zymography) and of murine u-PAR (PCR) (Grimaldi et al., 1986). The constructs used to transfect the cells with human wild-type or mutated u-PAR have been described (Roldan et al., 1990). LB6 clone 19 and NIH 3T3-u-PAR cells express about 500,000 and 300,000 receptors/cell, respectively on their surface as shown by direct binding analysis. LB6-D1HD3 and LB6-D1D2 express about 160.000 and 90.000 u-PAR molecules/cell on their surface, respectively. The dissociation constant for u-PA of wild type, clone 19, and D1HD3 clone was 2 nM, that of D1D2 clone was 8 nM (Riittinen et al., 1996). RSMC express u-PAR, u-PA and PAI-1 as shown by immunostaining methods. It was not possible to directly compare the levels of expression between rat and mouse cells, as the assays for the rat reagents are not at the same level of confidence. Human smooth muscle cells (AOSMC, CASMC) from the aortic and coronary arteries respectively, were cultured according to the supplier (Clonetics, Charlotte, N.C.). Human ATF was kindly provided by Dr. Jack Henkin (Abbott Park, Ill.). Soluble human UPAR may be prepared using the method of Tarui et al., 2001. Human active two-chain uPA was purchased from American Diagnostica. Mouse monoclonal anti-Stat1 antibody was from BD Biosciences, Transduction Laboratories (Lexington, Ky.). Mouse monoclonal anti-αvβ3 (LM 609) and anti-α5β1_antibodies, anti-mouse Ig rhodamine conjugated F(ab′)₂ fragment secondary antibody, and purified human αvβ3 and α5β1 integrins were bought from Chemicon (Temecula, Calif.). Non-specific monoclonal mouse IGG1K (MOPC-21), FITC-phalloidin, fP, forskolin, isobutyl-methyl-xanthine (IBMX) and Laminin (LN) were from Sigma. AG-490, and PD98059 were from Biomol (Plymouth Meeting, Pa.). As described (Degryse et al., 2001a), VN was purified from human plasma (Yatohgo et al., 1988). VN₄₀₋₄₅₉, the truncated form of human VN has been described (Okumura et al., 2002). Peptides D2A, and D2A-Ala derived from the sequence of domain 2 of human uPAR. Peptide D2A corresponds to the original human uPAR sequence ₁₃₀IQEGEEGRPKDDR₁₄₂, while in peptide D2A-Ala two glutamic acids were changed into two alanines giving the following sequence: IQEGAAGRPKDDR. Peptide D2B has the same aminoacid composition as D2A but a reverse sequence RDDKPRGEEGEQI.

### Chemotaxis Assay

Chemotaxis assay was performed as described (Degryse et al., 1999, 2001a, 2001b) with modified Boyden chambers. Filters (5 μm pore size, Neuro Probe, Gaithersburg, Md.) were treated with collagen I (100 μg/ml) and fibronectin (10 μg/ml, Roche).≈40-50,000 cells in serum-free DMEM were added to the upper well and the chemoattractants tested added to the lower well. When present, antibodies, or inhibitors were added in both wells. After overnight migration at 37° C., cells remaining on the upper surface of filters were scraped off and filters were fixed in methanol and stained in a solution of 10% (w/v) crystal violet in 20% (v/v) methanol. The experiments were performed at least twice in triplicate and the results, expressed as fold over control, are the mean ±SD of the number of cells counted in ten high power fields per filter. Random cell migration, i.e. migration in the absence of chemoattractant, was given the arbitrary value of 100%.

### Immunofluorescence Microscopy

As previously described (Degryse et al., 1999; 2004) 15,000-25,000 cells (20-40% confluence), were seeded on glass coverslips in 2 cm² well, cultured for 24 hours in DMEM plus 10% FCS, washed with PBS and cultured for another 24 hours without FCS. After stimulation, RSMC were fixed for 20 min at RT with 3% paraformaldehyde, 2% sucrose in PBS pH 7.5, washed 3 times with PBS-BSA 0.2%, permeabilized with 20 mM hepes pH 7.4, 300 mM saccharose, 50 mM NaCl, 3 mM MgCl₂, 0.5% (v/v) Triton X-100 for 3 min at 4° C., and washed 3 times with PBS-BSA 0.2%. RSMC were incubated in PBS-BSA 2% for 15 min at 37° C., then with anti-Stat1 primary antibodies for 30 min at 37° C., washed 3 times with PBS-BSA 0.2%, and further incubated with PBS-BSA 2% for 15 min at 37° C. Cells were stained with the secondary TRITC-antibody and with FITC-phalloidin for visualization of filamentous actin, for 30 min at 37° C. DAPI (4′,6-diamidino-2-phenylindole, Roche) was used to label the nucleus. Finally, after 3 washes with PBS-BSA 0.2%, one with distilled water, coverslips were mounted with 20% (w/v) Mowiol. Fluorescence Photographs were taken with a Zeiss Axiophot microscope

To quantify the activation of Stat1, random pictures were taken and nuclei positively stained for Stat1 were counted and expressed in percent.

### ¹²⁵I-VN Cell Binding

VN was iodinated using Iodogen (Pierce Chemical Co.) at a specific activity of 30-60×10⁶ cpm/mg. Briefly, 10 μg of VN dissolved in 0.1 M tris pH 7.6, 0.1% (v/v) Tween 80, were incubated with 0.5 μCi ¹²⁵Iodine for 15 minutes at room temperature, in a volume of 100 μl. The reaction was stopped by adding 1 ml of stop/elution buffer, 0.1 M tris pH 8.1, 0.1% (v/v) Chaps and 50 μl of N-acetyl tyrosine (10 mg/ml in 0.1 M tris pH 7.4). Labeled VN was separated from unbound ¹²⁵Iodine by gel chromatography in PD-10 column (Pharmacia) in the stop/elution buffer. SDS-PAGE followed by autoradiography revealed a 65-75 kDa doublet.

Cells were seeded in 96-well culture dishes treated overnight with 1.5% gelatin in PBS pH 7.4, grown to confluence in DMEM plus 10% FCS, then serum-starved for 24 hours in DMEM. Total binding was determined by incubating the cells on ice for 90 minutes in binding buffer (DMEM containing 0.1% (w/v) BSA and 10 mM Hepes pH 7.4) plus 0.5 nM ¹²⁵I-VN. Nonspecific binding was measured by competition with a 100 fold excess of unlabeled VN. At the end of the incubation time, the cells were washed 3 times with ice-cold binding buffer and once with ice-cold PBS, then lysed in a solution of 1% SDS, 1% Triton X-100 and the associated radioactivity counted. Cell number was determined in 6 wells per lines cultured in parallel, and the results were standardized as cpm/10⁵ cells.

### Pull-Down Assay

Soluble uPAR (suPAR) was radioiodinated (as for VN, see above) at a specific activity of 1.5×10⁵ cpm/ng. A mix composed of 2 μg of either purified αvβ3 or α5β1 integrin, and 2 g of ¹²⁵I-suPAR was incubated for 4 hours at 4° C. in the absence (control) or in the presence of 4 μg of uPA with or without 5 μg of either D2A or D2A-Ala peptide in binding buffer, (RPMI plus 0.02% BSA, 10 mM HEPES pH 7.4, total volume 300 μl). Complexes were immunoprecipitated with 3 μg of anti-αvβ3 (LM609) or anti-α5β1 monoclonal antibody, and 60 μl of protein G-agarose beads (Amersham Biosciences), Beads were washed three times in binding buffer, extracted in reducing sample buffer, and proteins were fractionated by SDS-PAGE. Gels were analyzed by autoradiography and densitometry. Results are presented as percentage of relative density units normalized to uPA alone.

### Statistical Analysis

Statistical analysis was performed with the Prism software using Student's t-test for pairwise comparisons of treatments, or an ANOVA model for the evaluation of treatments with increasing doses of a reagent.

### Example 1

### The Migratory Effect of VN Correlates with uPAR Expression

We have investigated the functionality of the integrin αvβ3-uPAR complex by examining the chemotactic effect of VN on mouse cells transfected or not with human uPAR cDNA.

NIH3T3 and LB6 murine cells express murine u-PAR: they were compared to their transfected counterparts NIH3T3-uPAR and LB6 clone 19 bearing respectively 300,000 and 500,000 human receptors/cell. In these two cell models, VN has chemotactic activity in both transfected and untransfected cells; however, an increased migratory response to VN was observed in cells transfected with uPAR and thus expressing a higher number of receptors (Table 1). At the dose of 5 μg/ml, VN-directed cell migration of LB6 clone 19 cells was enhanced 3.3 fold when compared to the migratory response of untransfected LB6 cells (Table 1). The difference between transfected and parental cells was highly significant (p values <0.001). Furthermore, VN migration-promoting effect was also enhanced by the expression of full-length uPAR in NIH-3T3 and human HEK-293 cells that are naturally devoid of uPAR. Table 1 shows that in transfected HEK-293-uPAR cells, the chemotactic effect of VN was 2-fold higher than in untransfected HEK-293 cells. Overall, these data show that u-PAR expression can affect the migratory response to VN, in agreement with a direct role of uPAR in VN-induced chemotaxis.

### Example 2

### VN-Directed Cell Migration does not Depend on VN Binding to uPAR but on the Presence of Intact uPAR on the Cell Surface

Since VN binds both integrins and uPAR; (Hoyer-Hansen 1997), the increased VN-dependent chemotaxis in uPAR overexpressing cells might be due to an increase of VN-binding sites. To investigate this possibility, we tested pools of LB6 and NIH3T3 cells transfected with either D1HD3-uPAR or D1D2-uPAR, mutated forms of human u-PAR lacking either domain 2 or 3 respectively, which are still able to bind human u-PA, pro-u-PA or ATF. (Riittinen et al., 1996). Since binding requires an intact u-PAR, these mutants are not expected to bind VN (Hoyer-Hansen 1997; Yebra, Goretzki et al. 1999) (Sidenius 2000). As expected, VN bound to LB6 clone 19 cells and poorly, but equally, to cells expressing the mutated forms of u-PAR (FIG. 1a).

We used these mutants to investigate the relationship between VN-dependent cell migration and VN binding to the cells. VN failed to stimulate the chemotaxis of NIH3T3 or LB6 cells even at 10 μg/ml (FIG. 1b), when these cells were transfected with D1HD3-uPAR. This effect was clone-independent because pools of transfected cells were used rather than clones. The fact that the migratory response to VN was totally absent in D1HD3 cells is surprising since VN stimulated 2.5 fold chemotaxis even in untransfected parental LB6 cells (Table 1). The D1HD3-uPAR mutant may therefore have a dominant negative effect on VN chemotaxis, possibly interfering with endogenous, murine uPAR. The results with the D1D2-uPAR mutant were different. Although LB6-D1D2 cells displayed lower sensitivity to low doses of VN compared to LB6 clone 19 cells (not shown), they still responded to VN with an about four-fold maximal stimulation at 1 μg/ml (FIG. 1b). In addition, cells transfected with D1D2-uPAR still responded to human ATF in chemotaxis assay, while D1HD3 and LB6 did not (FIG. 1c). Thus, in line with our previous report that VN migratory signal did not depend upon binding to uPAR (Degryse et al., 2001a), these data shows that the stimulation of chemotaxis by VN did not require its interaction with uPAR. To further confirm this observation, we used VN₄₀₋₄₅₉, a form of VN lacking the uPAR-binding SMB domain and hence unable to bind UPAR (Okumura et al., 2002). VN₄₀₋₄₅₉ stimulated cell migration as well as full-length VN (FIG. 1d), confirming that VN did not induce cell migration by binding to uPAR. These results suggest that VN induces cell migration by binding to its own integrin receptors. This conclusion is strengthened by our previous observation that anti-αvβ3 antibodies inhibited the chemotactic effect of VN (Degryse et al., 2001a, and see FIG. 3b). However, since anti-uPAR antibodies also inhibited VN-dependent chemotaxis (Degryse et al., 2001a), UPAR must be also directly involved. Therefore, uPAR involvement in the migration-promoting effect of VN might be due to a lateral interaction with the integrins (Simon 1996; Wei 1996). Since human wild-type u-PAR and D1D2-uPAR enhanced the effect of VN while a mutant lacking domain 2 prevented VN-induced cell migration (FIG. 1b), we hypothesized that domain 2 of UPAR might be directly involved in an interaction with an integrin. A region of domain 2, rich in charged amino acid residues and hence likely to be exposed, attracted our attention.

### Example 3

### D2A, a Peptide Derived from the Sequence of Domain 2 of Human uPAR, has Chemotactic Activity

To investigate this last hypothesis, we tested the effect of a synthetic peptide, D2A, ₁₃₀IQEGEEGRPKDDR₁₄₂, derived from the sequence of domain 2 of human UPAR. Interestingly, peptide D2A per se was able to stimulate migration of LB6-D1HD3 cells with a maximal 2-fold increase at 10 pM (FIG. 2a). Importantly; the addition of this peptide restored the response to VN of the otherwise non-responsive LB6-D1HD3 cells (FIG. 2a).

### Example 4

### Peptide D2A Stimulates Cell Migration Through αvβ3—, not uPAR-, Dependent Signaling Pathway

We also tested the effect of peptide D2A in a well-characterized cell system, the rat smooth muscle cells (RSMC) (Degryse et al, 1999, 2001a,b,c). These cells possess uPAR and αvβ3, and migrate in response to both uPA and VN challenge (Degryse et al., 1999; 2001a,c).

FIG. 2b shows that peptide D2A dose-dependently stimulated migration of RSMC. A maximum was obtained at 1 pM with a 3-fold increase of migration. However, the chemotactic effects of D2A and VN were not additive since the combination of optimal doses of peptide D2A (1 pM) and VN (1 μg/ml) did not further increase cell migration (FIG. 1d). Similar results were obtained with a combination of VN₄₀₋₄₅₉, and D2A (FIG. 1d). In contrast, the chemotactic effects of either uPA, or of peptides derived from the uPAR linker region (Fazioli et al., 1997), were additive with VN (data not shown) (Degryse et al., 2001a). Thus, these data suggest that peptide D2A and uPA use different molecular mechanisms.

We previously showed that uPA and VN synergized in RSMC chemotaxis because they activated different signaling pathways (Degryse et al., 2001a). Therefore, we pharmacologically investigated the pathways involved in D2A chemotaxis. Both VN and D2A chemotactic effects were blocked by increasing the intracellular cAMP concentration with a combination of IBMX and forskolin (FIG. 3a). We previously reported that this combination had no inhibitory effect on pro-uPA-induced cell migration (Degryse et al., 2001a). Neither VN- nor D2A-induced cell migration were blocked by the MEK inhibitor, PD98059 (FIG. 3b), which completely inhibited pro-uPA-induced cell migration, actin cytoskeleton reorganization, and ERK translocation into RSMC nucleus, but had no effect on VN chemotaxis (Degryse et al., 2001a). In addition, AG-490, a specific inhibitor of the Jak kinases, inhibited both D2A and VN chemotaxis in RSMC showing that these kinases are involved in mediating both chemotactic signals (FIG. 3c). Moreover, both VN- and D2A-induced cell migration appear to require αvβ3: indeed, they were inhibited by LM 609, a monoclonal antibody against αvβ3 enlightening the importance of this particular integrin in the regulation of both D2A and VN chemotaxis (FIG. 3d).

It is well known that chemoattractants induce the reorganization of actin cytoskeleton concomitantly with cell motility (Cooper, 1991). Using RSMC, we have previously functionally connected induction of cell migration and cytoskeleton reorganization (Degryse et al., 1999, 2001a,b), and conversely we have also correlated the lack of chemotactic effect with the absence of cytoskeleton reorganization (Degryse et al., 2001c). Thus, as a chemoattractant, D2A should also affect cell shape and actin cytoskeleton organization. To observe the influence of D2A or VN on cell morphology, actin cytoskeleton and Stat1 distribution, subconfluent cultures of serum-starved RSMC were stimulated with 1 pM peptide D2A or 1 μg/ml VN for 30 minutes at 37° C. Cells were triple-labeled with FITC-phalloidin, Dapi, and primary anti-Stat1 antibodies followed by TRITC-secondary antibodies to visualize the actin cytoskeleton, the nucleus, and Stat1 respectively. Unstimulated cells kept at 37° C. for 30 minutes represented the control conditions. FIG. 4 shows that most RSMC under control conditions exhibited numerous stress fibers and a nonpolarized cell shape. After 30 minutes of stimulation with D2A, a complete change of morphology and cytoskeleton organization occurred. RSMC had an elongated, polarized morphology reflecting the spatial rearrangement of the actin cytoskeleton. Semi-ring structures of actin and membrane ruffling were present at the leading part of the cell. Actin filaments were also observed flanking the nucleus and in the dragging trail. As expected (Degryse et al., 2001a), VN induced similar changes. Since the latent cytoplasmic transcription factor Stats are the main downstream substrates of the Jak kinases, we investigated the effects of D2A and VN on Stat1, one member of the Stat family of cytoplasmic proteins with roles as signal messenger and transcription factors. Intracellular localization of Stats proteins correlates with their activation state as cytoplasmic Stats are inactive, and translocate into the nucleus once activated (Aaronson and Horvath, 2002). In unstimulated control RSMC, Stat1 was mainly cytoplasmic but D2A treatment induced its translocation into the nucleus (FIG. 4). VN reproduced this effect on Stat1 localization (FIG. 4).

These results show that peptide D2A stimulates cell migration through activation of intracellular signaling pathways that are similar to the αvβ3-dependent pathways and different from the uPA-uPAR-FPRL1-dependent signaling pathways. These data also suggest that D2A mimics the interaction of uPAR with αvβ3 and hence activates this integrin-dependent signaling pathway supporting the idea that the epitope harbored by peptide D2A may be involved in uPAR-αvβ3 interaction.

### Example 5

### Peptide D2A Disrupts uPAR-αvβ3 Complexes

Since uPAR and αvβ3 form complexes on the cell surface (Myohanen et al., 1993; Xue et al., 1997; Czekay et al., 2003), we investigated the effects of peptide D2A on these complexes using a cell-free assay involving uPA-dependent co-immunoprecipitation of iodinated suPAR and unlabeled recombinant αvβ3. FIG. 5a shows that addition of uPA caused a nearly 10-fold increase in the amount of suPAR coimmunoprecipitated by antibodies against αvβ3 (lane 1 v. lane 2). In the presence of uPA plus D2A (lane 3), the amount of coimmunoprecipitated suPAR was reduced by approximately 50% compared to uPA alone. To demonstrate that these effects of D2A were not restricted to a single integrin, we repeated these experiments using α5β1 that also have been shown to interact with uPAR (Wei et al., 2001). Once again, similar results were obtained (FIG. 5b) showing that D2A can also prevent suPAR-β5β1 co-immunoprecipitation. These data suggest that peptide D2A contains an epitope of uPAR which is involved in uPAR-integrin interactions.

### Example 6

### Identification of Essential Chemotactic Residues of peptide D2A

To determine the chemotactic epitope present in D2A, we compared the chemotactic activity of peptides D2A and D2B that share the same amino acids composition but with a reversed sequence. Both peptides were equally active in stimulating migration of RSMC (FIG. 6a). This result suggested the presence of a similar epitope in both D2A and D2B peptides. Examination of their respective sequence revealed that these two peptides share a common GEEG sequence. A chemotactic activity of the GEEG sequence would explain why the reverse D2A sequence, D2B, was still chemotactic. To validate this hypothesis, we introduced changes in D2A sequence substituting the two glutamic acids for two alanines, giving a GAAG sequence. This new peptide, named D2A-Ala, was then compared to both D2A and D2B peptides using the chemotaxis assay. Under these conditions, peptide D2A-Ala failed to stimulate cell migration (FIG. 6a). These data suggest that the GEEG sequence of both D2A and D2B peptides was responsible for their chemotactic activity.

### Example 7

### D2A-Ala Inhibits VN-Induced Cell Migration

We further tested D2A-Ala by investigating its effects on VN-induced migration. Surprisingly, we found that, unlike D2A, peptide D2A-Ala completely abrogated the chemotactic effect of VN on RSMC (FIG. 6b). This result was confirmed by using primary cultures of human smooth muscle cells from the coronary artery (CASMC) and from the aorta (AoSMC). Again, D2A-Ala totally inhibited VN-induced migration of CASMC and AoSMC showing that D2A-Ala also blocked migration of human cells (FIGS. 6c,d). Thus, D2A-Ala behaves as an inhibitor of VN-induced cell migration.

To determine the IC₅₀ of this new inhibitor, we tested increasing concentrations of D2A-Ala on VN-stimulated RSMC, starting with doses as low as 1 fM. FIG. 7a shows that peptide D2A-Ala inhibits VN-induced cell migration in a dose-dependent manner. D2A-Ala is active in the fentomolar range. An effect is already visible at 10 fM, a complete inhibition was obtained at 1 pM with an IC₅₀ of approximately 10-20 fM

We also tested the effect of D2A-Ala on suPAR-αvβ3 interaction in vitro in the presence of uPA. As shown in FIG. 5a, lane 4, D2A-Ala also prevented uPAR-αvβ3-co-Immunoprecipitation suggesting that peptide D2A-Ala can still interact with the integrin αvβ3. However, D2A-Ala did not promote the change of morphology and reorganization of actin cytoskeleton typical of motile cells (FIG. 4). D2A-Ala also failed to activate Stat1 and its subsequent translocation into the nucleus (FIG. 4; 7b). Furthermore, D2A-Ala inhibited VN-induced translocation of Stat1 (FIG. 4; 7b). These data indicate that D2A-Ala interacts with the integrin αvβ3 in an antagonistic way preventing the transduction of a chemotactic signal. The exact molecular, mechanism is presently unknown but will be the focus of future experiments.

### Example 8

### D2A-Ala is a General Inhibitor of Integrin-Dependent Cell Migration

Since integrin αvβ3 mediates the chemotactic effect of VN, and since uPAR has been shown to interact with a wide diversity of integrins, we investigated the idea that D2A-Ala might also inhibit migration induced by other extracellular matrix (ECM) proteins such as FN and LN. Indeed, FN and LN are known to bind and act through different integrins, including integrin α5β1. D2A-Ala inhibited both FN- and LN-induced migration showing that it has a broad inhibitory effect (FIG. 8). These data suggest that D2A-Ala can block migration induced through different integrins. D2A-Ala also inhibited the uPA-induced co-immunoprecipitation of suPAR and α5β1, but to a much lesser extent (FIG. 5b). Even though the co-immunoprecipitation assay in vitro is very artificial, nevertheless the mutation introduced does not drastically prevent the uPAR-integrin complex-disrupting activity of this peptide.

In conclusion, these data show that D2A-Ala is a general inhibitor of extracellular matrix-induced, integrin-dependent, cell migration, and that it is active at very low concentrations, in the fM-pM range. In addition, D2A-Ala can disrupt at least some of the complexes uPAR forms with various integrins.

### Example 9

### The Migration-Promoting Effect of Peptide D2A does Require the Presence of uPAR on the Cell Surface

LB6-D1D2 cells are less sensitive to VN than LB6 clone 19 cells expressing full-length uPAR, in particular at very low concentrations (FIG. 1b). This suggests that other epitopes, maybe in domains 1 or 3, of uPAR may also be involved in the migratory signal induced by VN. This would not disagree with the dominant negative effect of D1HD3. We wondered therefore whether the effects of peptides D2A and D2A-Ala required the expression of uPAR on the cell surface. We compared HEK-293-uPAR and their parental counterpart that do not express uPAR (Resnati 2002) in their response to D2A and D2A-Ala. When we assayed the direct chemotactic effects, we observed that D2A elicited a strong effect on HEK-293-uPAR cells, but failed to stimulate the migration of HEK-293 cells (Table 2). This result indicates that uPAR expression is a pre-requisite for the D2A chemotactic effect. As expected, D2A-Ala did not induce migration in either HEK-293 or HEK-293-uPAR cells (Table 2). VN also induced chemotaxis in HEK-293 cells, and this effect was increased in the HEK-293 uPAR cells (Table 2). Also in this case, D2A did not modify the response to VN in neither cells. However, D2A-Ala was still capable of blocking VN chemotaxis even in the HEK-293 cells that do not express uPAR (Table 2, FIG. 6). Thus, while D2A needs uPAR expression to stimulate migration, the inhibitor D2A-Ala can inhibit VN chemotaxis even in the absence of uPAR.

In conclusion, these data show that the agonistic effect of D2A and the antagonistic effect of D2A-Ala use two slightly different mechanisms, which require the presence of uPAR on the cell surface for the former but not for the latter. Therefore the sequence of peptide D2A contains both “interaction” and “signaling” information and the two can be dissociated. Overall, the data reported here reinforce the notion that uPAR is a very important regulator of integrins signaling activity.

### Example 10

### Pleiotropic Inhibition of Chemotaxis by D2A-Ala and GAAG: Effect on EGF— and UTP-Dependent Chemotaxis in Rat Smooth Muscle Cells and in HT1080 Human Fibrosarcoma Cells

In Boyden chamber assays, D2A-Ala and GAAG inhibit not only VN-dependent and uPA-dependent chemotaxis (as reported above), but also EGF— and UTP-dependent chemotaxis. The effect is dose-dependent. Notice that the EGF-dependent and the UTP-dependent chemotaxis work through receptors (EGF-receptor and P2Y2 receptor, respectively), that are different among themselves and are different from those employed by uPA (uPAR) and VN (αvβ3). We conclude that these two inhibitors are general inhibitors of chemotaxis.

### Example 11

### D2A-Ala and GAAG Inhibit the in vitro Invasiveness of Human HT1080 Fibrosarcoma Cells

As cancer cells malignancy largely depends on its ability to invade and metastasize, we have tested whether D2A-Ala and GAAG also inhibit the invasiveness of cancer cells, using a Matrigel assay. As shown in FIG. 10, Panel a, the invasion observed after 24 hours at 37° C. in the presence of D2A or GEEG is similar to the level of HT1080 invasion induced by urokinase (uPA) or vitronectin (VN), which have been used as positive control. We have found that these same optimal concentrations of 1-10 pM also led to the maximum migration of mouse LB6 tumoral cells and normal rat smooth muscle cells (RSMC) suggesting a common mechanism spanning among various cell types and species.

In contrast, D2A-Ala and GAAG peptides have no effects and the numbers of cells that invaded through matrigel are similar to the value observed in control conditions. Similar data have been obtained after 7 days of invasion. The peptides D2A and GEEG, like uPA, VN and FN, stimulate the Matrigel invasiveness of HT1080 cells. The effect is dose-dependent reaching a peak around 1 pM (FIG. 10, Panel b). On the other hand, D2A-ala and GAAG inhibit the VN- and uPA-induced invasiveness at about the same concentration (FIG. 11, Panels a and b). D2A-ala and GAAG also inhibit FN-induced invasion at the same concentration (FIG. 12). Similar data are obtained after 7 days of invasion (with VN) or 2 days of invasion (with FN). We conclude that D2A-Ala and GAAG peptides inhibit invasion dependent on multiple integrins.

However, they also inhibit EGF— and insulin-induced invasiveness, albeit at a higher concentration (100 pM) (FIG. 13, Panels a and b).

This data confirms that D2A and GAAG are stimulators of uPA-, VN-, FN- and Ln-dependent chemotaxis and act through different integrins.

D2A-Ala and GAAG inhibit not only uPA-, VN-, FN-, and Ln-induced chemotaxis and cancer cell invasion, but also chemotaxis and invasion induced via different receptors (for example, uPAR, integrins, EGF-R, IR, PY2P-receptor).

The data thus further confirms that D2A-Ala and GAAG are general inhibitors of migration and invasion.

### Example 12

### Effects of Peptides of the D2A Family on Chemotaxis of the Human Hematopoietic Stem Cell Line KG-1 Cells

KG-1 cells are human acute myeloid leukemia cells derived from the bone marrow of a 59-year old Caucasian male with erythroleukemia that developed into acute myeloid leukemia. The line has characteristic of hematopoietic stem cells. This line has been isolated by Koeffler and Golde (1978; Science 200: 1153-1154). Cells are cultured in suspension in RPMI medium plus 10% FCS.

Chemotaxis assay of non adherent cells was performed as described (Degryse et al., 2001; FEBS lett. 505: 249-254) using modified Boyden micro-chamber with the exception that the filter was not treated. 50,000 cells were added in the top well in serum-free RPMI medium and allowed to migrate for 1 hour at 37° C. The molecules to be tested as chemoattractant were diluted in serum-free RPMI medium and added in the lower well of the Boyden chamber. Experiments were performed in triplicate. Results are the mean ±SD of the number of cells counted in five high power fields. Migration in the absence of chemoattractant is referred to as 100%.

FIG. 14 shows that D2A peptide stimulated migration of KG-1 cells. Its shorter version, the GEEG peptide was also chemotactic. Importantly, the D2B peptide that also harbors a GEEG motif (in fact, D2B has the same amino-acid composition as D2A but a reversed sequence) also promotes the migration of KG-1 cells. In contrast, Scr. D2A (that has the same amino-acid composition as D2A but a scrambled sequence), a scrambled version of peptide D2A that does not possess a GEEG epitope, was not chemotactic.

The D2A-Ala peptide and its shorter version the GAAG peptide, which both exhibit a GAAG epitope instead of a GEEG, motif, were not chemotactic (FIG. 1). However, both peptides inhibited vitronectin (VN)-induced chemotaxis of KG-1 cells.

In conclusion, these results confirm and extend the data previously obtained with other cell lines to hematopoietic stem cell lines. As expected, only peptides (D2A, D2B and GEEG) that harbor the GEEG epitope were chemotactic. D2A-Ala and GAAG peptides exhibiting the GAAG motif were not chemotactic. Furthermore, D2A-Ala and GAAG peptides blocked VN-induced chemotaxis of KG-1 cells and behaved as inhibitor of integrin-mediated migration.

All publications mentioned in the above specification are herein incorporated by reference. Various modifications and variations of the described methods and system of the invention will be apparent to those skilled in the art without departing from the scope and spirit of the invention. Although the invention has been described in connection with specific, preferred embodiments, it should be understood that the invention as claimed should not be unduly limited to such specific embodiments. Indeed, various modifications of the described modes for carrying out the invention which are apparent to those skilled in molecular biology or related fields are intended to be within the scope of the following claims.

TABLE 1
Effect of overexpression of u-PAR
on VN-dependent cell migration

| | Expression of | | |
|---|---|---|---|
| Cells | human u-PAR | No addition | VN |
| LB6 | — | 100 ± 2.7 | 274 ± 3.8 |
| LB6 clone 19 | Wild-type uPAR | 165 ± 4.7 | 906 ± 22.8 |
| NIH 3T3 | — | 100 ± 2.4 | 222.7 ± 4.8 |
| NIH 3T3-uPAR | Wild-type uPAR | 134.2 ± 4.0 | 397 ± 11.1 |
| HEK-293 | — | 100± | 199.1 |
| HEK-293-uPAR | Wild-Type uPAR | 87.1 | 394.3 |
| LB6-D1HD3 | Mutant | 100 ± 2.0 | 102 ± 5.0 |
| | D1HD3-uPAR | | |
| LB6-D1D2 | Mutant | 100 ± 3.5 | 280 ± 6.0 |
| | D1D2-uPAR | | |
| NIH 3T3-D1HD3 | Mutant | 100 ± 3.0 | 100 ± 4.0 |
| | D1HD3-uPAR | | |

Data are expressed as % of control ± SD. VN concentrations were 5 μg/ml for LB6, LB6 clone 19, LB6-D1HD3 and LB6-D1D2, and 1 μg/ml for NIH 3T3, NIH 3T3-uPAR, NIH 3T3-D1HD3, HEK-293 and HEK-293-uPAR cells.

TABLE 2
Effects of uPAR expression on the agonistic and antagonistic effects
of peptides D2A and D2A-Ala, respectively, on cell migration.

| | HEK-293 | HEK-293-uPAR |
|---|---|---|
| Control | 100.00 ± 24.52 | 92.28 ± 33.80 |
| D2A (1 pM) | 106.98 ± 11.78 | 325.48 ± 14.42 |
| D2A-Ala (1 pM) | 110.19 ± 26.63 | 108.72 ± 17.37 |
| VN (1 μg/ml) | 210.84 ± 26.95 | 417.25 ± 4.64 |
| VN (1 μg/ml) + D2A (1 pM) | 190.23 ± 35.24 | 400.88 ± 17.41 |
| VN (1 μg/ml) + D2A-Ala (1 pM) | 94.50 ± 19.58 | 108.18 ± 13.90 |

Data are expressed as % of control ± SD.

### REFERENCES

Aaronson, D. S., and C. M. Horvath. (2002). A road map for those who don't know JAK-STAT. Science 296:1653-1655.
Aguirre Ghiso, J. A., Kovalski, K. and Ossowski, L. (1999). “Tumor dormancy induced by downregulation of urokinase receptor in human carcinoma involves integrin and MAPK signaling.” J. Cell Biol. 147(1): 89-104.
Appella, E., Robinson, E. A., Ulrich, S. J., Stoppelli, M. P., Corti, A., Cassani, G. and Blasi, F. (1987). The receptor-binding sequence of urokinase. A biological function for the growth-factor module of proteases. J. Biol. Chem. 262: 4437-4440.
Blasi, F. (1999). The urokinase receptor. A cell surface, regulated chemokine. APMIS. 107:96-101.
Blasi, F., and P. Carmeliet. (2002). uPAR: a versatile signaling orchestrator. Nat. Rev. Mol. Cell. Biol. 3:932-943.
Bugge, T., Suh T T, Flick M J, Daugherty C C, Romer J, Solberg H, Ellis V, Dano K and Degen J L. (1995). The receptor for urokinase-type plasminogen activator is not essential for mouse development or fertility. J. Biol. Chem. 270: 16886-16894.
Chapman, H. A. (1997). Plasminogen activator, integrins, and the coordinated regulation of cell adhesion and migration. Curr. Opin. Cell Biol., 9, 714-724.
Chavakis, T., S. M. Kanse, F. Lupu, H. P. Hammes, W. Muller-Esterl, R. A. Pixley, R. W. Colman, and K. T. Preissner. (2000). Different mechanisms define the antiadhesive function of high molecular weight kininogen in integrin- and urokinase receptor-dependent interactions. Blood 96:514-522.
Chavakis, T., S. M. Kanse, A. E. May, and K. T. Preissner. 2002. Haemostatic factors occupy new territory: the role of the urokinase receptor system and kininogen in inflammation. Biochem. Soc. Trans. 30:168-173.
Colman, R. W., R. A. Pixley, S, Najamunnisa, W. Yan, J. Wang, A. Mazar, and K. R. McCrae. (1997). Binding of high molecular weight kininogen to human endothelial cells is mediated via a site within domains 2 and 3 of the urokinase receptor. J. Clin. Invest. 100:1481-1487.
Cooper, J. A. (1991). The role of actin polymerization in cell motility. Annu. Rev. Physiol. 53:585-605.
Czekay, R. P., K. Aertgeerts, S. A. Curriden, and D. J. Loskutoff. (2003). Plasminogen activator inhibitor-1 detaches cells from extracellular matrices by inactivating integrins. J. Cell Biol. 160:781-791.
Degryse, B. (2003). Is uPAR the centre of a sensing system involved in the regulation of inflammation? Curr. Med. Chem.-Anti-Inflammatory & Anti-Allergy Agents. 2:237-259.
Degryse, B., and M. de Virgilio. (2003). The nuclear protein HMGB1, a new kind of chemokine? FEBS Lett. 553:11-17.
Degryse, B., M. Resnati, S. A. Rabbani, A VIIIa, F. Fazioli, and F. Blasi. (1999). Src-dependence and pertussis-toxin sensitivity of urokinase receptor-dependent chemotaxis and cytoskeleton reorganization in rat smooth muscle cells. Blood 94:649-662.
Degryse, B., S. Orlando, M Resnati, S. A. Rabbani, and F. Blasi. (2001a). Urokinase/urokinase receptor and vitronectin/alpha(v)beta(3) integrin induce chemotaxis and cytoskeleton reorganization through different signaling pathways. Oncogene 20:2032-2043.
Degryse, B., T. Bonaldi, P. Scaffidi, S. Muller, M. Resnati, F. Sanvito, G. Arrigoni, and M. E. Bianchi. (2001b). The high mobility group (HMG) boxes of the nuclear protein HMG1 induce chemotaxis and cytoskeleton reorganization in rat smooth muscle cells. J. Cell Biol. 152:1197-1206.
Degryse, B., C. F. Sier, M. Resnati, M. Conese, and F. Blasi. (2001c). PAI-1 inhibits urokinase-induced chemotaxis by internalizing the urokinase receptor. FEBS Lett. 505:249-254.
Degryse, B., J. G. Neels, R. P. Czekay, K. Aertgeerts, Y. Kamikubo, and D. J. Loskutoff. (2004). The low density lipoprotein receptor-related protein is a motogenic receptor for plasminogen activator inhibitor-1. J. Biol. Chem. 279:22595-22604.
Deng, G., Curriden, S. A., Wang, S., Rosenberg, S, and Loskutoff, D. J. (1996). Is plasminogen activator inhibitor-1 the molecular switch that governs urokinase receptor-mediated cell adhesion and release? J Cell Biol 134: 1563-71.
Dewerchin, M., Nuffelen A V, Wallays G, Bouche A, Moons L, Carmeliet P, Mulligan R C,. Collen D. (1996). Generation and characterization of urokinase receptor-deficient mice. J. Clin. Invest. 97: 870-878.
Estreicher, A., Wohlwend A., Belin D., Schleuning, W. D. and Vassalli, J. -D. (1989). Characterization of the cellular binding site for the urokinase-type plasminogen activator. J. Biol. Chem. 264: 1180-1189.
Fazioli, F., M. Resnati, N. Sidenius, Y. Higashimoto, E. Appella, and F. Blasi. (1997). A urokinase-sensitive region of the human urokinase receptor is responsible for its chemotactic activity. EMBO J. 16:7279-7286.
Grimaldi, G., P. Di Fiore, E. K. Locatelli, J. Falco, and F. Blasi. (1986). Modulation of urokinase plasminogen activator gene expression during the transition from quiescent to proliferative state in normal mouse cells. EMBO J. 5:855-861.
Gyetko, M. R., S. Sud, T. Kendall, J. A. Fuller, M. W. Newstead, and T. J. Standiford. (2000). Urokinase receptor-deficient mice have impaired neutrophil recruitment in response to pulmonary Pseudomonas aeruginosa infection. J. Immunol. 165:1513-1519.
Gyetko, M. R., S. Sud, J. Sonstein, T. Polak, A. Sud, and J. L. Curtis. (2001). Antigen-driven lymphocyte recruitment to the lung is diminished in the absence of urokinase-type plasminogen activator (uPA) receptor, but is independent of uPA. J. Immunol. 167:5539-5542.
Gyetko, M. R., Todd, R. F. 3rd, Wilkinson, C. C., and Sitrin, R. G. (1994). The urokinase receptor is required for human monocyte chemotaxis in vitro. J. Clin. Invest. 93: 1380-1387.

Hoyer-Hansen, G., Behrendt, N., Ploug, M., Dano, K. and Preissner, K. T. (1997). The intact urokinase receptor is required for efficient vitronectin binding: receptor cleavage prevents ligand interaction. FEBS Lett 420(1): 79-85.
Kanse, S. M., C. Kost, O. G. Wilhelm, P. A. Andreasen, and K. T. Preissner. (1996). The urokinase receptor is a major vitronectin-binding protein on endothelial cells. Exp. Cell Res. 224:344-353.
Kjoeller, L. a. Hall, A. (2001). Rac mediates cytoskeletal rearrangements and increased cell motility induced by urokinase-type plasminogen activator receptor binding to vitronectin. J. Cell Biol. 152: 1145-1157.
Koshelnick, Y., Ehart, M., Hufnagl, P., Heinrich, P. C. and Binder, B. R. (1997). Urokinase receptor is associated with the components of the JAK:STAT1 signaling pathway and leads to the activation of this pathway upon receptor clustering in the human kidney epithelial tumor cell line TCL-598J. Biol. Chem. 272: 28563-28567.
Liu, D., Aguirre-Ghiso, J. A., Estrada, Y. and Ossowski, L. (2002). EGFR is a transducer of the urokinase receptor initiated signal that is required for in vivo growth of a human carcinoma. Cancer Cell 1: 445-457.
May, A. E., S. M. Kanse, L. R. Lund, R. H. Gisler, B. A. Imhof, and K. T. Preissner. (1998). Urokinase receptor (CD87) regulates leukocyte recruitment via beta 2 integrins in vivo.
J. Exp. Med. 188:1029-1037.
Myohanen, H. T., R. W. Stephens, K. Hedman, H. Tapiovaara, E. Ronne, G. Hoyer-Hansen, K. Dano, and A. Vaheri. (1993). Distribution and lateral mobility of the urokinase-receptor complex at the cell surface. J, Histochem. Cytochem. 41:1291-1301.
Nguyen, D. H., I. M. Hussaini, and S. L. Gonias. (1998). Binding of urokinase-type plasminogen activator to its receptor in MCF-7 cells activates extracellular signal-regulated kinase 1 and 2 which is required for increased cellular motility. J. Biol. Chem. 273:8502-8507.
Nguyen, D. H., Webb, D. J., Catling, A. D., Song, Q., Dhakephalkar, A., Weber, M. J., Ravichandran, K. S, and Gonias, S. L. (2000). Urokinase-type plasminogen activator stimulates the Ras/Extracellular signal-regulated kinase (ERK) signaling pathway and MCF-7 cell migration by a mechanism that requires focal adhesion kinase, Src, and Shc. Rapid dissociation of GRB2/Sps-Shc complex is associated with the transient phosphorylation of ERK in urokinase-treated cells. J. Biol. Chem. 275: 19382-19388.
Okumura, Y., Y. Kamikubo, S. A. Curriden, J. Wang, T. Kiwada, S. Futaki, K. Kitagawa, and D. J. Loskutoff. (2002). Kinetic analysis of the interaction between vitronectin and the urokinase receptor. J. Biol. Chem. 277:9395-9404.
Ossowski, L., G. Clunie, M. T. Masucci, and F. Blasi. (1991). In vivo paracrine interaction between urokinase and its receptor: effect on tumor cell invasion. J. Cell Biol. 115:1107-1112.
Ossowski, L., and J. A. Aguirre Ghiso. (2000). Urokinase receptor and integrin partnership: coordination of signaling for cell adhesion, migration and growth. Curr. Opin. Cell Biol. 12:613-620.
Ploug, M. (2003). Structure-Function Relationship in the interaction between urokinase-type plasminogen activator and its receptor. Curr. Pharmac. Design, 9, 1499-1528.
Porter, J., Hogg N. (1998). Integrins take partners: cross-talk between integrins and other membrane receptors. Trends Cell Biol. 8: 390-396.
Preissner, K., Kanse, S. M. and May, A. E. (2000). Urokinase receptor: a molecular organizer in cellular communication. Curr. Opin. Cell Biol. 12: 621-628.
Resnati, M., M. Guttinger, S. Valcamonica, N. Sidenius, F. Blasi, and F. Fazioli. (1996). Proteolytic cleavage of the urokinase receptor substitutes for the agonist-induced chemotactic effect. EMBO J. 15:1572-1582.
Resnati, M., Pallavicini, I., Wang, Y. M., Oppenheim, J., Serhan, C. N., Romano, M. and Blasi, F. (2002). The fibrinolytic receptor for urokinase activates the G protein-coupled chemotactic receptor FPRL1/LXA4R. Proc. Natl. Acad. Sci. U.S.A. 99:—1359-1364.
Ridley, A. J., H. F. Paterson, C. L. Johnston, D. Diekmann, and A. Hall. (1992). The small GTP-binding protein rac regulates growth factor-induced membrane ruffling. Cell 70:401-410.
Ridley A J, S. M., Burridge K, Firtel R A, Ginsberg M H, Borisy G, Parsons J T, Horwitz A R. (2003). Cell migration: integrating signals from front to back. Science, 302. 1704-1709.
Riittinen, L., P. Limongi, M. P. Crippa, M. Conese, L. Hernandez-Marrero, F. Fazioli, and F. Blasi. (1996). Removal of domain D2 or D3 of the urokinase receptor does not affect ligand affinity. FEBS Lett. 381:1-6
Rijneveld, A. W., M. Levi, S. Florquin, P. Speelman, P. Carmeliet, and T. van Der Poll. (2002). Urokinase receptor is necessary for adequate host defense against pneumococcal pneumonia. J. Immunol. 168:3507-3511.
Roldan, A. L., M. V. Cubellis, M. T. Masucci, N. Behrendt, L. R. Lund, K. Dano, E. Appella, and F. Blasi. (1990). Cloning and expression of the receptor for human urokinase plasminogen activator, a central molecule in cell surface, plasmin dependent proteolysis. EMBO J. 9:467-474.
Sidenius, N. and Blasi, F. (2000). Domain 1 of the urokinase receptor (uPAR) is required for uPAR-mediated cell binding to vitronectin. FEBS L. 470: 40-46.
Simon, D. I., Rao, N. K., Xu, H., Wei, Y., Majdic, O., Ronne, E., Kobzik, L. and Chapman, H. A. (1996). Mac-1 (CD11b/CD18) and the urokinase receptor (CD87) form a functional unit on monocytic cells. Blood 88: 3185-3194.
Simon, D. I., Y. Wei, L. Zhang, N. K. Rao, H. Xu, Z. Chen, Q. Liu, S. Rosenberg, and H. A. Chapman (2000). Identification of a urokinase receptor-integrin interaction site. Promiscuous regulator of integrin function. J. Biol. Chem. 275:10228-10234.
Tarui, T., Mazar, A. P., Cines, D. B., Takada, Y. (2000). Urokinase receptor (uPAR/CD87) is a ligand for integrin and mediates cell-cell interaction. J. Biol. Chem. 276: 3983-3990.
Tarui, T., A. P. Mazar, D. B. Cines, and Y. Takada. (2001). Urokinase-type plasminogen activator receptor (CD87) is a ligand for integrins and mediates cell-cell interaction. J. Biol. Chem. 276:3983-3990.
Tarui, T., Andronicos N, Czekay R P, Mazar A P, Bdeir K, Parry G C, Kuo A, Loskutoff D J, Cines D B, Takada Y. (2003). Critical role of integrin alpha 5 beta I in urokinase (uPA)/urokinase receptor (uPAR, CD87) signaling. J Biol. Chem. 278: 29863-29872.
Vacca, A., Ria R, Presta M, Ribatti D, Iurlaro M, Merchionne F, Tanghetti E, Dammacco F. (2001). Alpha(v) beta(3) integrin engagement modulates cell adhesion, proliferation, and protease secretion in human lymphoid tumor cells. Exp Hematol. 29: 993-1003.
Waltz, D. A., and H. A. Chapman. (1994). Reversible cellular adhesion to vitronectin linked to urokinase receptor occupancy. J. Biol. Chem. 269:14746-14750.
Wei, Y., D. A. Waltz, N. Rao, R. J. Drummond, S. Rosenberg, and H. A. Chapman. (1994). Identification of the urokinase receptor as, an adhesion receptor for vitronectin. J. Biol. Chem. 269:32380-32388.
Wei, Y., J. A. Eble, Z. Wang, J. A. Kreidberg, and H. A. Chapman. (2001). Urokinase receptors promote beta1 integrin function through interactions with integrin alpha3beta1. Mol. Biol. Cell. 12:2975-2986.
Wei, Y., M. Lukashev, D. I. Simon, S. C. Bodary, S. Rosenberg, M. V. Doyle, and H. A. Chapman. (1996). Regulation of integrin function by the urokinase receptor. Science 273:1551-1555.
Xue, W., 1. Mizukami, R. F., 3rd Todd, H. R. Petty. (1997). Urokinase-type plasminogen activator receptors associate with beta1 and beta3 integrins of fibrosarcoma cells: dependence on extracellular matrix components. Cancer Res. 57:1682-1689.
Xue, W., Kindzelskii, A. L., Todd, R. F., 3rd and Petty, H. R. (1994). Physical association of complement receptor type 3 and urokinase-type plasminogen activator receptor in neutrophil membranes. J Immunol 152(9): 4630-40.
Yatohgo, T., M. Izumi, H. Kashiwagi, and M. Hayashi. (1988). Novel purification of vitronectin from human plasma by heparin affinity chromatography. Cell Struct. Funct. 13:281-292.
Yebra, M., L. Goretzki, et al. (1999). Urokinase-type plasminogen activator binding to its receptor stimulates tumor cell migration by enhancing integrin-mediated signal transduction. Exp Cell Res 250(1): 231-240.
Yebra, M., Parry, G. C. N., Stromblad, S., Mackman, N., Rosenberg, S., Mueller, B. M. and Cheresh, D. A. (1996). Requirement of receptor-bound urokinase-type plasminogen activator for integrin alphavbeta5-directed cell migration. J Biol Chem 271(46): 29393-29399.
de Paulis A, Montuori N, Prevete N, Fiorentino I, Rossi F W, Visconte V, Rossi G, Marone G, Ragno P. Urokinase induces basophil chemotaxis through a urokinase receptor epitope that is an endogenous ligand for formyl peptide receptor-like 1 and like 2. J. Immunol. 2004 Nov. 1; 173(9):5739-48.
Selleri C, Montuori N, Ricci P, Visconte V, Carriero M V, Sidenius N, Serio B, Blasi F, Rotoli B, Rossi G, Ragno P. Involvement of the Urokinase-type plasminogen activator receptor in hermatopoietic stem cell mobilization. Blood; 2004 Oct. 19; [Epub ahead of print]
Nykjaer, A., Moller, B., Todd, R. F., 3rd, Christensen, T., Andreasen, P. A., Gliemann, J. & Petersen, C. M. (1994) J. Immunol. 152, 505-516.
Sidenius, N., Olsen, J. E., Sier, C. F. M. Blasi, F., Ullum, H. Increased plasma levels of soluble urokinase receptor in HIV infected patients are highly correlated with survival. Blood, 96, 4091-4095, 2000.

1. A polypeptide which inhibits cell migration, cell adhesion, cell proliferation and/or cell differentiation and which comprises the amino acid motif GAAG (SEQ ID NO: 2).

2-14. (canceled)

15. The polypeptide according claim 1, wherein the polypeptide comprises or consists of the amino acid motif GAAG (SEQ ID NO: 2) and which polypeptide is derivable from the urokinase receptor (uPAR).

16. The polypeptide according claim 1 comprising or consisting of the amino acid sequence IQEGAAGRPKDDR (SEQ ID NO: 3) or RDDKPRGAAGEQI (SEQ ID NO: 4).

17. (canceled)

18. The polypeptide according to claim 1 comprising or consisting of domain 2 of the uPAR in which the amino acid residues at positions 34 and 35 of the wild type sequence are changed from glutamic acid to alanine or its reverse or a fragment thereof.

19. A polypeptide according claim 1 in a modified form.

20. (canceled)

21. A polynucleotide encoding for the polypeptide of claim 1.

22. An expression vector comprising a polynucleotide encoding for the polypeptide of claim 1.

23. (canceled)

24. A pharmaceutical composition comprising the polypeptide of claim 1, together with a pharmaceutically acceptable carrier, excipient or diluent.

25. A method for treating or controlling a disease or condition associated with cell migration, cell adhesion, cell proliferation and/or cell differentiation comprising administering an effective amount of the polypeptide of claim 1 to a patient in need of the same.

26. A method for treating or controlling cancer or metastasis or the invasive ability of a cancer cell comprising administering an effective amount of the polypeptide of claim 1 to a patient in need of the same.

27.-28. (canceled)

29. The method according to claim 26 wherein the cancer is an adenocarcinoma, a leukemia, lymphoma or a myeloma.

30. A method for treating or controlling angiogenesis, fibrosis of tissue, inflammation, an immune disorder, epithelial cell hyperplasia, an infectious disease or a disease associated therewith comprising administering an effective amount of the polypeptide of claim 1 to a patient in need of the same.

31-61. (canceled)

62. A method of identifying an agent that is a modulator of uPAR, integrin, such as αvβ3, α5β1 α3β1, or VN, FN, LN, EGF-R, P2Y2, insulin-R activity comprising: determining uPAR, integrin, such as αvβ3, α5β1 α3β1, or VN, FN, LN, EGF-R, P2Y2, insulin-R activity respectively in the presence and absence of said agent; comparing the activities observed; and identifying said agent as a modulator by the observed differences in uPAR integrin, such as αvβ3, α5β1 or α3β1, VN, FN, LN, EGF-R, P2Y2, insulin-R activity (as appropriate) in the presence and absence of said agent; and wherein the method involves the use of the polypeptide of claim 1.

63. The method according to claim 62 further comprising preparing said agent.

64. The agent identifiable according to the method of claim 62

65. An antibody directed against the polypeptide of claim 1.

66. The method according to claim 62 further comprising preparing said agent.

67. A pharmaceutical composition comprising a polypeptide comprising or consisting of the amino acid sequence IQEGAAGRPKDDR (SEQ ID NO: 3) or RDDKPRGAAGEQI (SEQ ID NO: 4), together with a pharmaceutically acceptable carrier, excipient or diluent.

68. A method of treating or controlling a disease or condition associated with cell migration, cell adhesion, cell proliferation and/or cell differentiation comprising administering and effective amount of a polypeptide comprising or consisting of the amino acid sequence IQEGAAGRPKDDR (SEQ ID NO: 3) or RDDKPRGAAGEQI (SEQ ID NO: 4).

69. A method for treating or controlling cancer or metastasis of the invasive ability of a cancer cell comprising an effective amount of a polypeptide comprising or consisting of the amino acid sequence IQEGAAGRPKDDR (SEQ ID NO: 3) or RDDKPRGAAGEQI (SEQ ID NO: 4) to a patient in need of the same.

70. A method of treating or controlling angiogenesis, fibrosis or tissue, inflammation, an immune disorder, epithelial cell hyperplasia, an infectious disease or disease associated therewith comprising administering an effective amount of a polypeptide comprising or consisting of the amino acid sequence IQEGAAGRPKDDR (SEQ ID NO: 3) or RDDKPRGAAGEQI (SEQ ID NO: 4) or the pharmaceutical composition of claim 69 to a patient in need of the same.
